(19) United States
(12) Patent Application Publication
(10) Pub. No.: US 2014/0080115 A1
(43) Pub. Date: Mar. 20, 2014

(54) DEVICE AND METHOD FOR MONITORING THE PRESENCE, ONSET AND EVOLUTION OF PARTICULATES IN CHEMICALLY OR PHYSICALLY REACTING SYSTEMS

(71) Applicant: Wayne F. Reed, New Orleans, LA (US)

(72) Inventor: Wayne F. Reed, New Orleans, LA (US)

(73) Assignee: THE ADMINISTRATORS OF THE TULANE EDUCATIONAL FUND, New Orleans, LA (US)

(21) Appl. No.: 13/985,560

(22) PCT Filed: Feb. 14, 2012
(86) PCT No.: PCT/US2012/025041

§ 371 Date: Nov. 26, 2013

Related U.S. Application Data
(60) Provisional application No. 61/463,293, filed on Feb. 14, 2011.

(51) Int. Cl. G01N 33/44; C12Q 1/04; C12Q 3/00; B01D 35/147

(52) U.S. Cl. CPC G01N 33/442; B01D 35/1475; C12Q 1/04; C12Q 3/00
USPC 435/3; 210/132; 73/61.71; 422/73; 435/288.7; 435/34; 436/34

(57) ABSTRACT

A device for monitoring particulates includes a means for correlating measurements of pressure across at least one filter, flow rate of a sample through the filter, or a combination thereof to the properties of particulates in a solution. More particularly, the device can be used for monitoring particulates in a reacting system to provide signals to the user or control input to the reacting system to alter the course of the reaction according to a desired path.

### CROSS REFERENCE TO RELATED APPLICATIONS

Priority of my U.S. Provisional Patent Application Ser. No. 61/463,293, filed 14 Feb. 2011, incorporated herein by reference, is hereby claimed.

### STATEMENT REGARDING FEDERALLY SPONSORED RESEARCH

Not Applicable

### COMPACT DISC SUBMISSION

Not Applicable

### BACKGROUND OF THE INVENTION

1. Field of the Invention

The present invention relates to monitoring particulates in reacting systems. More particularly, the present invention relates to a means of measuring the presence and evolution of particulate matter, such as microgels, cross-linked polymers, and biological cells, that arise during chemical, biochemical, and physical reactions, or in bioreactors where bacteria or other living organisms build up as a particulate population and produce a desirable product, such as a food or pharmaceutical agent, or in processing of natural products where particulates such as microgels and starch and cellulosic fragments are present.

2. Description of Related Art

There is an industrial need for monitoring particulates in the polymer and natural products industries. In many chemically, biochemically, and physically reacting systems, and/or systems being non-reactively processed, certain types of particulate matter can be produced or decomposed. For example, in a polymerization reaction, particulates often form during the reaction that consist of physical microgels of the polymer being produced, or covalently cross-linked polymers, or microcrystals, or other types of aggregates, such as salt crystals. In processing natural products for example, e.g. extracting and separating polysaccharides from their precursors, microgels and ‘fines’ may be released into the process or waste streams. In general these particulates are undesirable, since they can compromise the quality of the end product; e.g. lead to irregular textures, substandard tensile strengths, difficulties in dissolution, cloudy product solutions, etc. Furthermore, the buildup of these particles can lead to fouling of large reactors which lead to costly clean-up and maintenance procedures, including reactor shutdowns and lost production time. Sometimes the buildup of particulates is desirable, such as in the case of polymerization reactions whose products precipitate out of the reacting solution.

A non-exhaustive list of other chemical reactions in which particulates can increase, decrease, or remain constant during reactions includes:

In many different types of polymerization reactions—those carried out in batch, in a semi-continuous or continuous way, under pressure, at ambient pressure, different temperatures, in bulk, in solvent, in multiple stages, in emulsions, inverse emulsions, micelles, suspensions, etc. particulate matter can form, such as crosslinked microgels, physically associated aggregates and microgels, microcrystals, latex particles, emulsion and oil droplets, salt crystals.

In emulsification of products, e.g. foodstuffs, particles can decrease in size as emulsion proceeds, and increase in size upon de-emulsification.

In bioreactors the microbial cell count can increase or decrease as a fermentation or other reaction dependent upon microbial metabolism proceeds.

In the production of biofuels particulate matter, often cellulosic, decreases in size and quantity as the raw biomass is processed into a pure fuel.

Upon blending polymeric materials micro-phase separation can occur with the concomitant production of polymeric aggregates and other particulates.

Certain multi-component systems, e.g. surfactants, metal ions and polymers in solution, will begin to aggregate and precipitate under certain environmental conditions, such as heat.

During a polymerization reaction salts or other non-polymeric species form microcrystals.

Dissolution of grains; e.g. a biopolymer, such as guar or pectin, has a broad grain size distribution, from microns to mm. As it dissolves in water the particulate population decreases.

Processing natural products. When separating out desired endproducts from intermediate products in a process stream, particulates will often be released. The particulates can be microgels of the material, undissolved fragments of cell walls and organelles, and other particulates often termed ‘fines’.

Most particulate monitoring is currently done by optical and scattering methods that are usually costly and difficult to maintain, especially in industrial settings. For example, dynamic light scattering (e.g. Brookhaven Instruments Corp BI-90 Plus) (Berne and Pecora, 1975) and Mie scattering (e.g. Malvern Mastersizer) (Kerker, 1969) are elegant but costly means of characterizing particulates. Further, they require clean, conditioned samples. While such clean samples may be available in the laboratory, they are not generally available directly from industrial reactors. Disc centrifugation with optical detection is a time-consuming method not easily adaptable to an online monitoring environment. Particle detectors that use optical occlusion, time of flight, and changes in dielectric constants also require clean, well-conditioned samples.

The Automatic Continuous Online Monitoring of Polymerization reactions (ACOMP) technology (Reed, 2003; Florenzano, et al., 1998—see my U.S. Pat. No. 6,653,150) provides a means of making absolute, continuous measurements of all the important characteristics of polymerization reactions, such as conversion, kinetics, and evolution of average composition, molecular weight and intrinsic viscosity distributions. One embodiment of ACOMP also allows simultaneous characterization of particulates, (Reed, 2010; Alb & Reed 2008—see my U.S. Pat. No. 7,716,969) e.g. emulsions and polymer latex particles, in addition to the polymers, by withdrawing two streams of reactor contents, and diluting one with a solvent that homogenizes the contents to yield polymer and monomer characteristics, and the other stream is diluted with a solvent (e.g. water) that preserves the emulsions. The particulates (emulsions and polymer latex particles) in this latter stream are then characterized by an optical means, such as Mie or dynamic light scattering, UV/visible absorption, turbidity, etc. However, use of these optical means contains the same limitations as discussed above.

Thus, there is a need for a device that provides a means of detecting and analyzing, in real-time, the presence, onset and evolution of particulates for an industrial setting.

Incorporated herein by reference are all patents, patent applications, patent application publications, and publications mentioned herein, including the following:

### U.S. PATENT DOCUMENTS

U.S. Pat. No. 6,052,184 April 2000 Reed 356/338
U.S. Pat. No. 6,618,144, Device and method of simultaneously measuring the light scattering from multiple liquid samples containing polymers and/or colloids
U.S. Pat. No. 6,653,150 B1, November 2003, Reed, Automatic mixing and dilution methods for online characterization of equilibrium and non-equilibrium properties of solutions containing polymers and/or colloids
US 2004/0004717 Reed, Wayne F., Jan. 8, 2004, Automatic mixing and dilution methods and apparatus for online characterization of equilibrium and non-equilibrium properties of solutions containing polymers and/or colloids
U.S. Pat. No. 7,716,969 B2 May 2010 Reed, et al. 73/61.71
US 2008/0008623 A1, Reed; Wayne F., Jan. 10, 2008, Automatic sampling and dilution apparatus for use in a polymer analysis system
US 2009/0306311 A1, Reed; Wayne F., Dec. 10, 2009, METHODS AND INSTRUMENTATION FOR DURING-SYNTHESIS MONITORING OF POLYMER FUNCTIONAL EVOLUTION
U.S. Pat. No. 4,550,591
U.S. Pat. No. 6,230,551
U.S. Patent Application 2010/0192679
U.S. Pat. No. 4,765,963
U.S. Patent Application 2011/0271739
U.S. Pat. No. 5,932,795
U.S. Pat. No. 7,224,455
U.S. Pat. No. 3,824,395
U.S. Pat. No. 7,162,057
U.S. Pat. No. 3,628,139

### OTHER PUBLICATIONS

B. Berne and R. Pecora, Dynamic Light Scattering, John Wiley, 1975.
M. Kerker, The Scattering of Light and Other Electromagnetic Radiation, Academic Press, 1969.
F. H. Florenzano, R. Strelitzki, and W. F. Reed, Macromolecules, 1998, 31, 7226-7238.
A. M. Alb, W. F. Reed, Macromolecules, 2008, 41, 2406-2414.

Incorporated herein by reference are all patents, patent applications, and patent application publications naming the present inventor as an inventor, and all publications naming the present inventor as an author.

While certain novel features of this invention shown and described below are pointed out in the annexed claims, the invention is not intended to be limited to the details specified, since a person of ordinary skill in the relevant art will understand that various omissions, modifications, substitutions and changes in the forms and details of the invention illustrated and in its operation may be made without departing in any way from the spirit of the present invention. No feature of the invention is critical or essential unless it is expressly stated as being “critical” or “essential.”

### BRIEF SUMMARY OF THE INVENTION

The present invention makes use of time-dependent filter behavior which is the essential notion of what the present inventor terms “filtrodynamics”—a mathematical analysis of detailed time dependent signatures of changing pressure (or flow) across slowly plugging filters to make physical interpretations of the particulate characteristics. Filtrodynamics is preferably used in the context of polymer manufacturing and processing. The present invention can include ACOMP, SMSLS, and other detector schemes with the filtrodynamic instruments.

The present invention can include measuring time dependent behavior and mathematically deducing particulate characteristics, with no need for knowledge of the pore sizes of a filter to get the number of particles. The present invention can include making continuous (or substantially continuous) measurements and a mathematical analysis of a time-dependent filter response to determine particulate properties. By substantially continuous the present inventor means, in the context of preferred embodiments of the present invention, at least 6 measurements per hour, more preferably at least 1 measurement per minute, and most preferably at least 20 measurements per minute.

The present invention typically deals with microscopic and nanoscopic particles that are not affected by gravity over the time scales of measurement and the associated reactions and processes. The present invention typically deals with plugging filters and measuring time-dependent flow or pressure signatures.

The present invention can use time-dependent pressure signals to make deductions about particle size distributions.

The present invention provides a device to monitor the state of particulates in chemically, biochemically, or physically reacting systems, and systems that are otherwise being processed. Such systems are normally in liquid phases such as solvents, emulsions, suspensions, etc. The device preferably comprises at least one non-optical sensor, which can give a time-dependent signal related to particulate build-up, on a filter in a reacting system that can be cleaned or changed with minimal operational downtime. The device is useful in gaining knowledge of the state of particulates preferably at each instant allowing understanding and control over the process. The device is especially useful for signaling the completion of a process and moving to a subsequent process, signaling a problem in a process and correcting the problem, signaling that there is contamination in the product, or signaling that a process is proceeding desirably.

The present invention provides a device that may include a sequence of filters that filter liquids, arranged in parallel, in series, or in some combination thereof. An object of the present invention is to measure the status of particulates in the system by measuring increasing resistance across one of more filters. The device may comprise a sequence of pressure sensors, flow rate sensors, or some combination thereof to gather data that can be related to a particular filter's resistance.

A signal feature of the invention is that the filtration system is used to diagnose properties of the process or reaction system, not to actually filter the contents of the system itself for purposes of cleaning or improving the reaction or process contents. In this context the invention will normally sample and pass through its filter-sensor system a very tiny fraction of the entire reactor or process content. As an example, not limiting in scope, the invention may withdraw and filter, for diagnostic purposes, on the order of 10 mL of reaction or process liquid from a reaction or process vessel containing liters to tens of thousands of liters or more during a one hour reaction or process.

Another important feature of the invention related to its status as a diagnostic instrument is that its filtration can protect other diagnostic or monitoring equipment downstream from its filter-sensor network. For example, while its main function is to diagnose the existence, level, or type of impurities in the reaction or process vessel, it may direct its filtered output stream to flow through other monitoring and diagnostic equipment downstream from it which requires filtered or conditioned sample streams. The invention may also use its diagnostics to diagnose itself, in that it can signal a changeover of filter-sensor flowpaths when certain pressure or flow criteria are met. In all these embodiments, the present invention is characterized by filtering a very small fraction of reaction or process liquids for the purpose of monitoring and diagnosis. Preferably and normally, much less than 1% of the reaction or process liquids are withdrawn for diagnostic purposes, such as less than 0.1%, more preferably less than 0.01%, even more preferably less than 0.001%, and most preferably less than 0.0001%. Withdrawals of 1% or 0.1% by volume might occur during a research and development lab reaction. Withdrawal of 0.000001% by volume might be more typical for a batch reactor. In the case of continuous reactors, there would be similar percentages of the continuous reactor flow rate, not of the volume. For batch and semi-batch reactions the withdrawals are expressed in volumetric %, whereas for continuous reactors the withdrawals are expressed in flow rate percentages. For a laboratory scale reaction with 500 ml this would represent 2% of the reactor content per hour. For an industrial scale reactor of 10,000 gallons, this would represent approximately 0.000026% per hour. Typical reactions of interest are on the scale of 0.5 to ten hours, although some reactions can be faster or slower than this. In the case of continuous reactors, a very small percentage of the continuous reactor flow rate would be diverted through the filtrodynamic system. For example, a continuous reactor with a flow rate of 10,000 gallons per hour would have approximately 0.000026% of that flow diverted throughout its continuous operation.

The present invention provides a device that may include a means to indicate if a reacting system is performing according to a desired path based on particulate number density or size.

The present invention provides a device that gives control capabilities to a reacting system based on the state of particulates and a desired system response. For example, if a desired system response requires a certain range of particulates for optimal operation, the present invention can change aspects of the reaction to meet this desired operation, or stop the reacting system, if necessary.

The control capabilities provided by the present invention can include any type of open loop or closed loop system used in R&D and industrial situations. For example, the present invention's output information concerning onset of particle formation, or density, or type, could be used to signal an operator to manually take actions that affect the reaction or process. In a closed loop system the present invention's output data could be used with an automatic system controller (such as are commonly provided by such companies as Honeywell, Johnson Controls, ABB, Foxboro Corp., Emerson Electric, Rockwell, etc.) to automatically make decisions and take actions that affect the reaction or process.

The present invention may provide a device with removable cartridges containing filters, such that filters can be manually or automatically changed if the system indicates that the filters are plugged or are becoming highly plugged with particulates.

The present invention provides a method for determining the state of particulates by determining the resistance of a filter in a reacting system or for characterizing particulates in non-reacting, equilibrium systems. Resistance can be determined by measuring pressure, flow rate, or some combination thereof of a fluid through a reacting system. The present invention provides a method of correlating particulate size and numbers with filter resistance by means of, for example, multiple filters having different pore sizes.

The present invention provides a method for signaling various states of particulates by determining the resistance on a filter or multiple filters in a reacting system.

The present invention provides a method of determining resistance by measuring the pressure difference across the filter, or the flow rate through the filter, or some combination thereof.

The present invention provides a method of determining filter resistance by measuring the pressure difference across the filter when the flow rate is constant.

The present invention provides a method of determining filter resistance by measuring the flow rate through a filter when the pressure difference across the filter is constant.

The present invention provides a method to measure particle size distribution in solutions with stable particulate populations, such as reaction end products and intermediate aliquots. The present invention provides a method to continuously measure particle size distributions in chemically and/or physically reacting solutions.

An object of this invention is to correlate filter resistance to particulate population and size either on an intermittent or continuous basis.

An object of this invention is to characterize particulate populations in non-reacting systems. For example, the present invention may characterize non-reacting solutions that have stable populations of particulates, such as end products of polymerization, or intermediate reaction aliquots which have been withdrawn and quenched. Such characterization includes determination of particle size distribution (PSD), a central focus of the particle sizing field, which is dominated by optically based technologies.

An object of the present invention is to characterize particulate populations in chemically and/or physically reacting solutions.

An object of the present invention is to use the time-dependent pressure signals across filters or networks of filters to make deductions concerning the presence or change of particulate populations in a reaction or process. For example, the rate at which pressure builds, or at which flow decreases, can be used to compute the particle density and size distribution in the reaction or process, and the rates at which these change. The invention will hence generate time-dependent pressure ‘signatures’ which can be analyzed to monitor and diagnose properties of particulates in reactions and processes. The mathematical form of these ‘signatures’ contains important information on both steady and changing particulate populations in reactions and processes.

Other objects and advantages of this invention will become readily apparent from the ensuing description.

### BRIEF DESCRIPTION OF THE DRAWINGS

FIG. 1 shows a representative example of the presence of particulates in a stable (non-reacting) multi-component polymer solution (a linear polymer in monochlorobenzene) leading to increasing pressure measured across a single filter, due to the gradual built up of particulates on a 0.5 micron filter through which a continuously diluted stream of the solution was pumped at constant flow rate.

FIG. 2 shows a representative network of n filters in parallel.

FIG. 3 shows a representative series network.

FIG. 4 shows representative filters in series, each with a pressure sensor and two-way diverter valve.

FIG. 5 illustrates a variation that can be used if the output line is feeding a detector train rather than going to waste.

FIG. 6 shows three different illustrations of pressure with respect to time.

FIG. 7 illustrates 1/P(t), which will yield polynomials in t that give a more striking signature that differentiates the three scenarios.

FIG. 8 shows another set of cases under constant flow Q₀ and where the sizes of particles remain constant.

FIGS. 9 and 10 shows the relationship between flow rate and time under constant pressure.

FIG. 11 shows one embodiment of the invention that enables a plugging-free operation via parallel flow paths in which the filter in each flow path has the same pore size.

FIG. 12 shows an embodiment of the invention in which the filters are in series, each with a pressure sensor and two-way diverter valve.

FIG. 13 illustrates an additional embodiment of the invention, in which the output line is feeding a detector train rather than going to waste.

FIG. 14 illustrates how an embodiment of the invention can be used for monitoring presence and evolution of particulates in a polymerization reactor.

FIG. 15 illustrates an exemplary ‘front end’ of an intermittently operated dilution instrument.

FIG. 16 illustrates an intermittent reverse flow used to purge and clean the filters.

FIG. 17 shows conceptual signals for reciprocal pressure vs. time for a series of 18 filters.

FIG. 18 shows nᵢ, which is a measurement of concentration, obtained from FIG. 17.

FIG. 19 shows the PSD obtained from the above nᵢ values of FIG. 18.

FIG. 20 shows the reciprocal pressure vs. time for 5 filters in series, filter 1 having the smallest pore size, and filter 5 having the largest.

FIG. 21 shows a conceptual set of pressure signals on filters 1, 2 . . . n during the hypothetical polymerization reaction.

FIG. 22 shows the build up of trans-filter pressure (in atmospheres) when a dilute solution of 2.0 micron latex spheres flows through a 0.45 PTFE Millipore filter of 13 mm diameter at 0.2 ml/min.

FIG. 23 shows pressure signals to be quite repeatable for multiple injections of the polysaccharide solution through a fresh 0.8 μm cellulose acetate filter on three consecutive runs.

FIG. 24 shows data fit by the equation form derived for a probabililstic filter (A/(B+exp(−xt)).

FIG. 25 shows 2 micron frit, gatekeeping effect, 0.2 ml/min).

FIG. 26 shows viscosity pressure correlation showing gatekeeping.

FIG. 27 shows 0.8 μm cellulose ester membrane filter with 2 mg/ml polysaccharide solution yielded no gatekeeping.

FIG. 28 shows 2 mg/ml polysaccharide, 0.8 Mic CE filter, no gatekeeping.

FIG. 29 shows 0.45 mic mixed cellulose esters, 2 mg/ml Guar1, 0.2 ml/min, no gatekeeping.

FIG. 30 shows raw data from a filtrodynamic arrangement consisting of two pressure transducers (Ashcroft Corp, type G1) measuring the transfilter pressure of two filters in series.

FIG. 31 is a valve schematic.

FIG. 32 shows how a single outlet flow can be had by joining the two unconnected outlets shown in the diagram with a two-way valve.

FIG. 33 shows series filters schematic example: random cut-off sequence for n=4 filters in series.

### DETAILED DESCRIPTION OF THE INVENTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

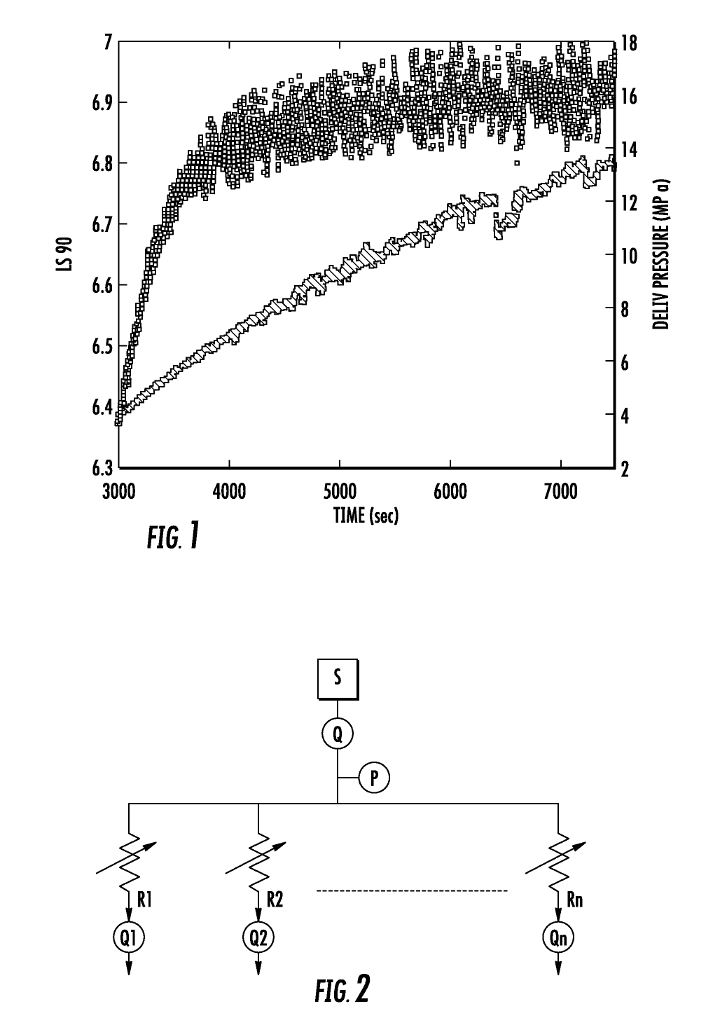

FIG. 1 shows a representative example of the presence of particulates in a stable (non-reacting) multi-component polymer solution (a linear polymer in monochlorobenzene) leading to increasing pressure measured across a single filter, due to the gradual built up of particulates on a 0.5 micron filter through which a continuously diluted stream of the solution was pumped at constant flow rate. In this case, the particulates were microcrystals of salt left over from the polymerization reaction that produced the polymers. In this application, the pressure signal across the filter was monitored to determine when the pressure was too high for safe system operation; i.e. use of pressure signals and attendant relief valves, servo-mechanisms, etc. have been used for centuries as means of insuring safe operating ranges and limits for pumps, heat engines, etc, but not for particulate analysis. It is noted that the pressure signal has an initial linear regime and then has a negative second derivative. This signature, not surprisingly, is closer to the types predicted for the probabilistic filters described below, where pores are not well defined and so any given particle has some probability of getting through the filter. The filter for this was a sintered metal frit filter, which acts as a short 3D filter, not a membrane, so the probabilistic filter nature of the pressure signal below is not surprising.

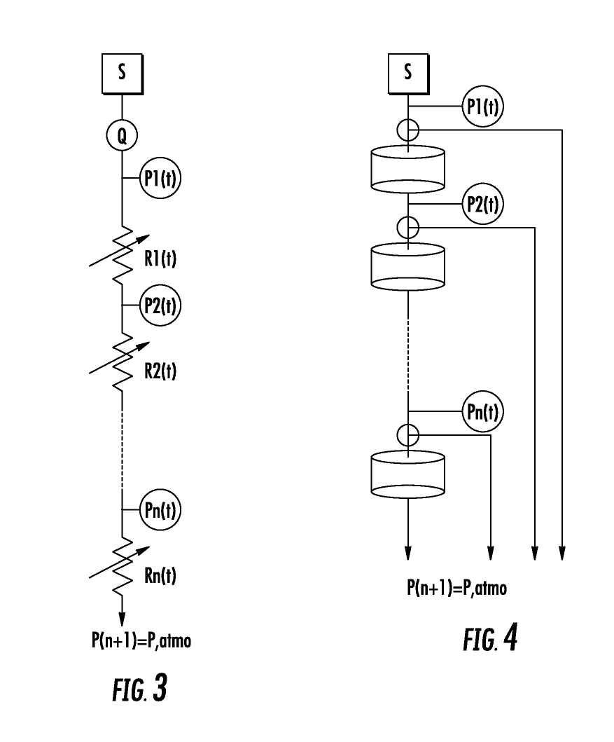

FIG. 2 shows a representative network of n filters in parallel.

FIG. 3 shows a representative series network.

FIG. 4 shows representative filters in series, each with a pressure sensor and two-way diverter valve. In position 1 the two way valve directs flow into the next filter, whereas in position 2 it is directed to waste.

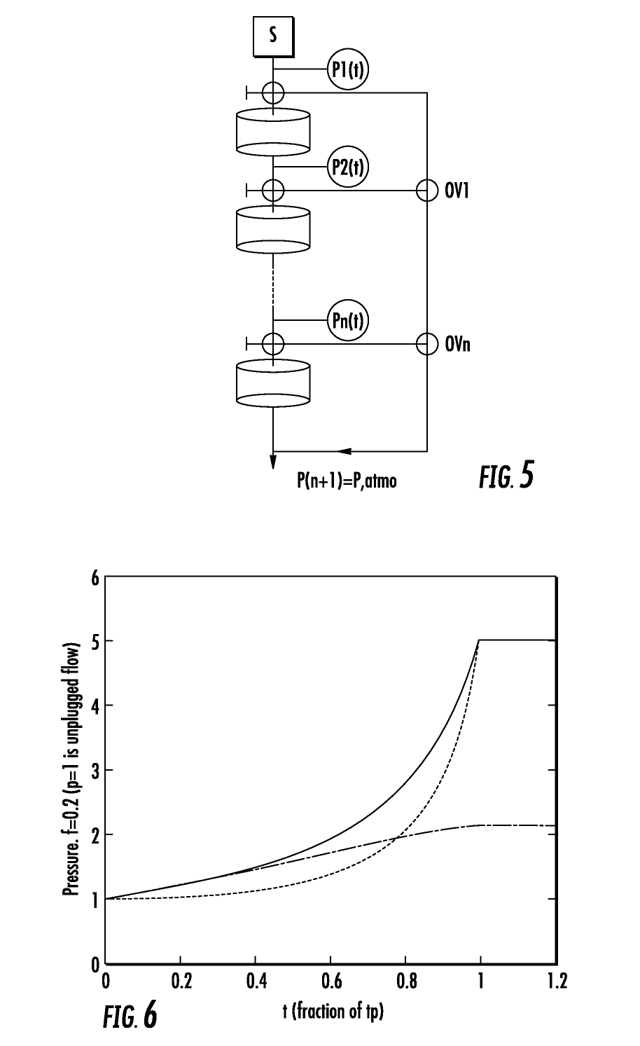

FIG. 5 illustrates a variation that can be used if the output line is feeding a detector train rather than going to waste. In this case a three way valve is used; in position 1 the two-way valve directs flow into the next filter, in position 2 it is directed to waste, and position 3 shuts off flow through the valve.

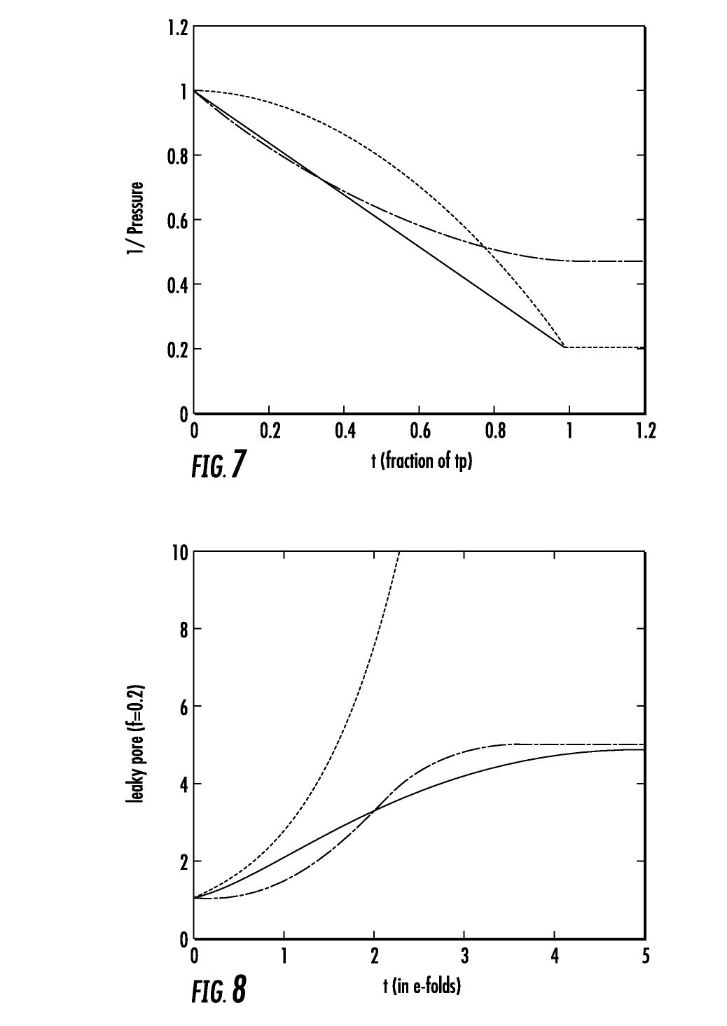

FIG. 6 shows three different illustrations of pressure with respect to time. In FIG. 6, time is expressed as a fraction of total time necessary to achieve complete blockage of all pores on the filter. The solid line is a model of pressure as a function of time. This line assumes that particulate concentration is constant. The dotted line models the pressure as a function of time in the case of a fluid with linearly increasing particulate concentration, as in the case of a reaction fluid where particulates are being formed as the reaction proceeded. The dashed line models the pressure, as a function of time, of a fluid with decreasing particulate concentration, as in the case of a reaction mixture with an initial concentration of particulates, and demonstrates decreased particulate concentration as the reaction proceeded. f is a ‘leakage factor’, whose value is 0 for a non-leaky particle/pore interaction and 1 when a trapped particle does not impede flow.

FIG. 7 illustrates 1/P(t), which will yield polynomials in t that give a more striking signature that differentiates the three scenarios. The solid line models a steady particulate population that yields a straight line for P(t) vs. t and the second derivative, d²P/dt²=zero. The dotted line models the case of increasing particulates d²P/dt²<0, and the dashed line models the case of decreasing particulates d²P/dt²>0. f is a ‘leakage factor’, whose value is 0 for a non-leaky particle/pore interaction and 1 when a trapped particle does not impede flow.

FIG. 8 shows another set of cases under constant flow Q₀ and where the sizes of particles remain constant. It is assumed that the rate of pore pluggage to degree f is proportional to the remaining amount of pore area, which yields an exponential decrease in total filter area. f is a ‘leakage factor’, whose value is 0 for a non-leaky particle/pore interaction and 1 when a trapped particle does not impede flow. The dotted line indicates a non-leaky filter (f=0). For cases where there is a leaky filter (f>0), then a plateau in pressure is reached where P_{final}/Pₒ=1/f. The dashed line indicates a linearly increasing population of particles.

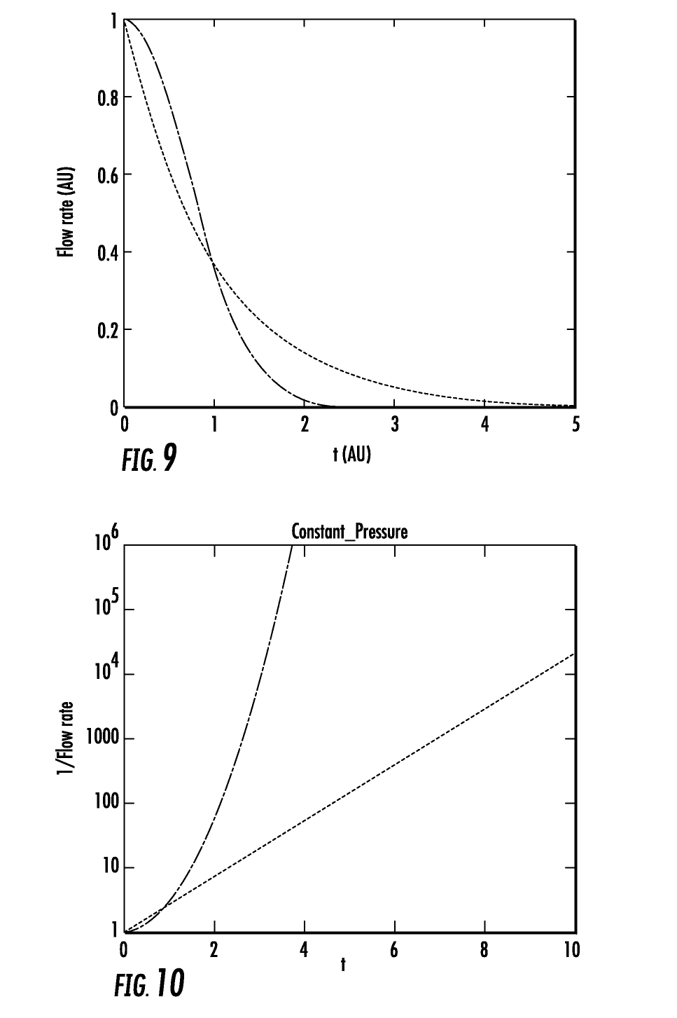

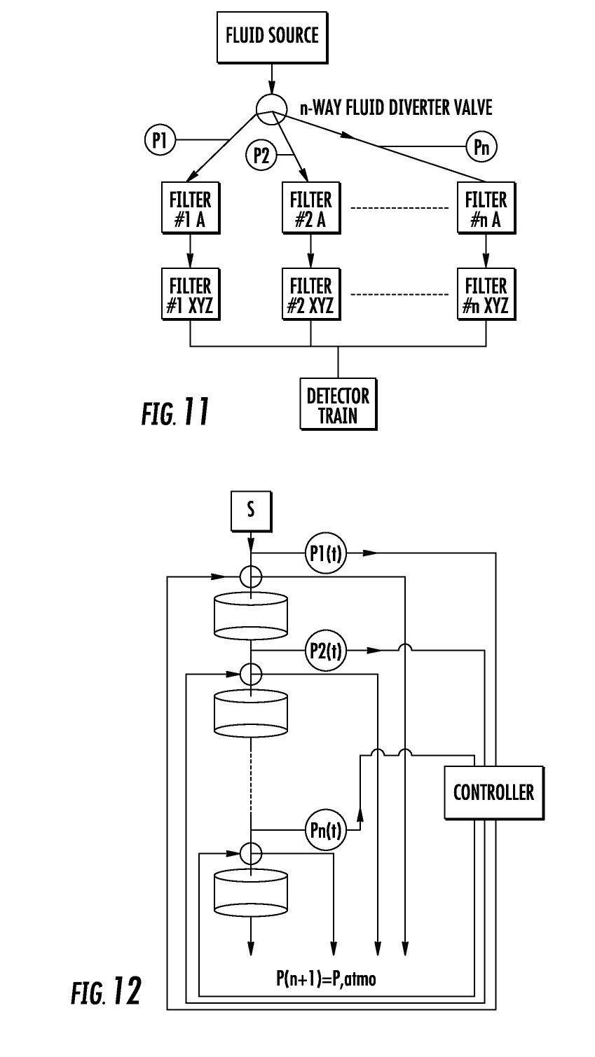

FIGS. 9 and 10 show the relationship between flow rate and time under constant pressure.

FIG. 11 shows one embodiment of the invention that enables a plugging-free operation via parallel flow paths in which the filter in each flow path has the same pore size.

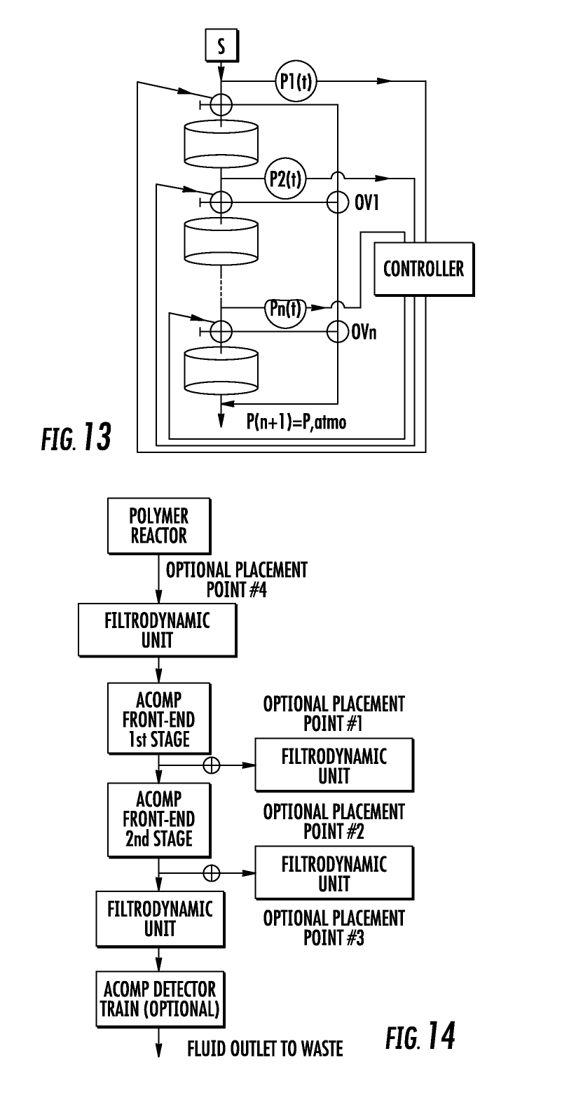

FIG. 12 shows an embodiment of the invention in which the filters are in series, each with a pressure sensor and two-way diverter valve. In position 1 the two-way valve directs flow into the next filter, whereas in position 2 flow is directed to waste.

FIG. 13 illustrates an additional embodiment of the invention, in which the output line is feeding a detector train rather than going to waste. In this case a three-way valve is used, where in position 1 the three-way valve directs flow into the next filter, in position 2 flow is directed to waste, and position 3 shuts off flow through the valve. The controller again has the same function as in the FIG. 12, except that it can now command each valve into three different positions, instead of two.

FIG. 14 illustrates how an embodiment of the invention can be used for monitoring presence and evolution of particulates in a polymerization reactor. The net contents of the above type embodiments, shown in FIGS. 2-5 and 11-13, are termed the ‘Filtrodynamic Unit’, and include filter networks, pressure and/or flow sensors, controller, and optional analysis means. In FIG. 14, the control and analysis means can be integrated into the ACOMP control and analysis platform. It should be noted that more than one filtrodynamic unit can be used in the figure; i.e. filtrodynamic units could be installed at more than one of the ‘optional’ sites shown in the figure.

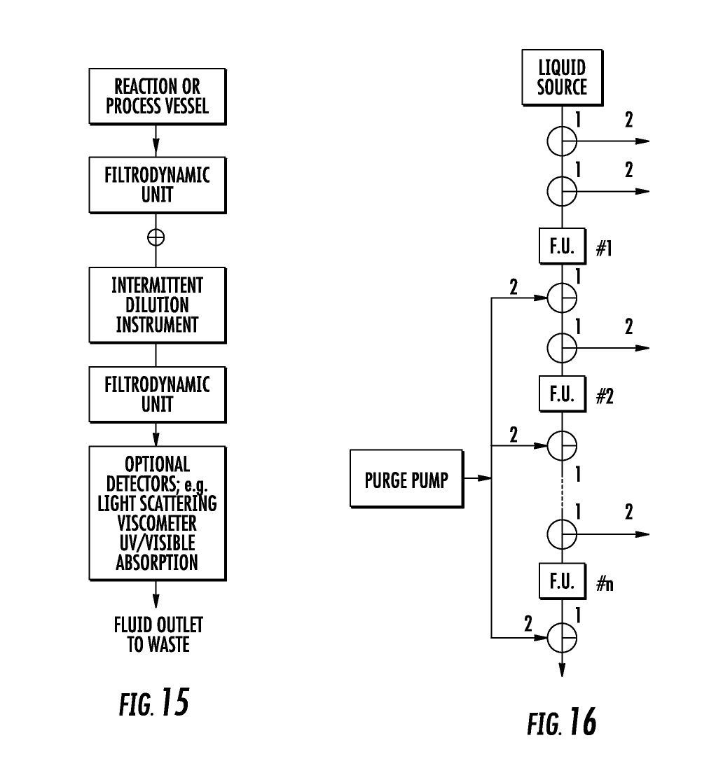

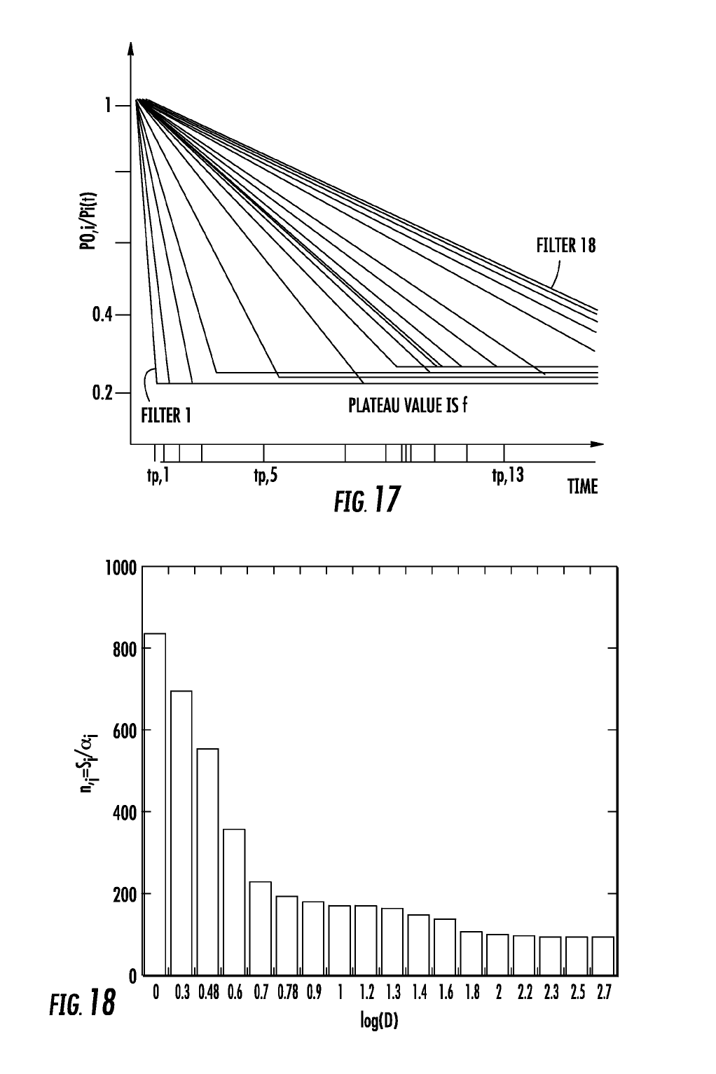

FIG. 15 illustrates an exemplary ‘front end’ of an intermittently operated dilution instrument. It makes withdrawals of reactor fluid at defined time intervals, and dilutes them in a mixing chamber(s) (not shown in FIG. 15, but can include, for example, the apparatus shown in FIG. 16 or 17 of my U.S. Pat. No. 6,653,150 B1), which diluted reactor liquid is then used to feed the Filtrodynamic Unit in the defined time intervals.

FIG. 16 illustrates an intermittent reverse flow used to purge and clean the filters.

FIG. 17 shows conceptual signals for reciprocal pressure vs. time for a series of 18 filters, where filter 1 (the last filter) has the smallest pore size and filter numbering increases with pore size up to filter 18 (the first filter in the series), which has the largest pore size. The pluggage time tₚ for each filter is shown in the vertical lines along the time axis, and a few are labeled for illustration; e.g. t_{p,1}. The reciprocal of the plateaus value for each filter is 1/fᵢ. Different plateau values are shown to indicate that different filters may have different leakage factors fᵢ.

FIG. 18 shows nᵢ, which is a measurement of concentration, obtained from FIG. 17. The absolute values of the slopes in FIG. 17 are used in computing nᵢ.

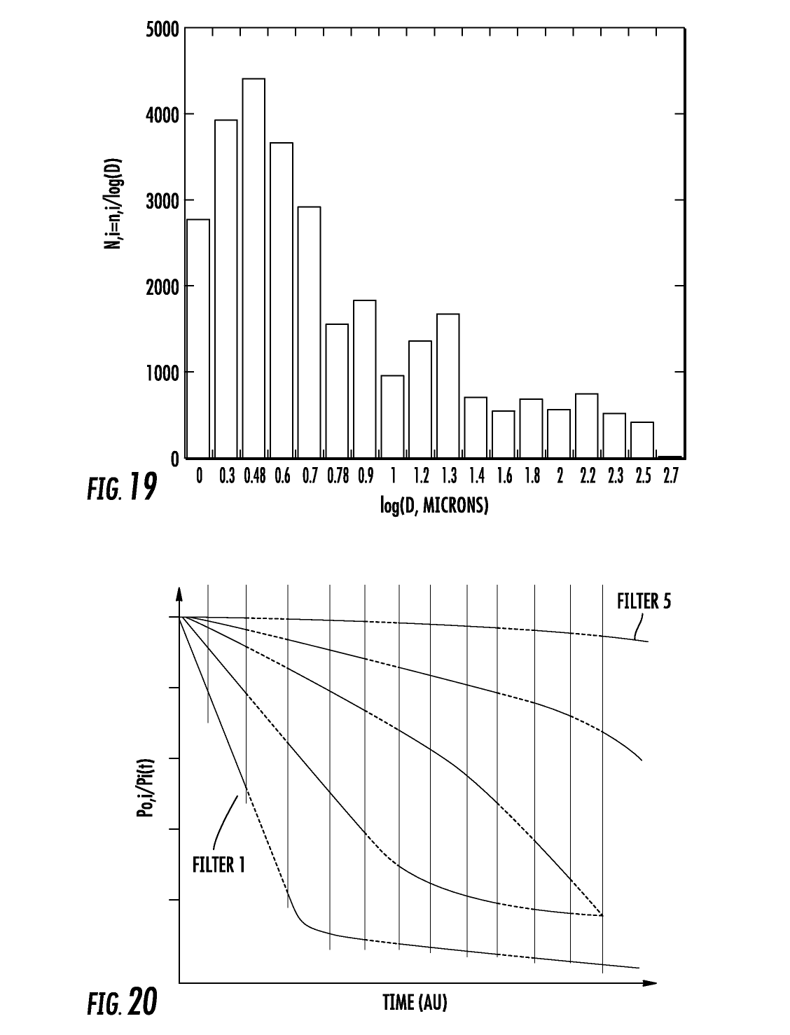

FIG. 19 shows the PSD obtained from the above n, values of FIG. 18. It shows the concentration (particles/cm³) in the size ranges shown on the logarithmic x-axis (microns). As in other particle sizing methodologies, the distribution below can be left as a histogram or, by well known methods, smoothed into a continuous function, fit to an assumed analytic form, etc.

FIG. 20 shows the reciprocal pressure vs. time for 5 filters in series, filter 1 having the smallest pore size, and filter 5 having the largest. The vertical lines show time intervals, Δt, into which the data can be broken. Over each interval a linear fit to the reciprocal pressure of each filter can be made from the slopes sᵢ, applied, to yield the PSD at each time interval k; N(Dᵢ,tₖ). In the graph the dotted line segments are examples of the linear fits that would be made over a few chosen intervals Δt.

FIG. 21 shows a conceptual set of pressure signals on filters 1, 2 . . . n during the hypothetical polymerization reaction. Filter 1 has the smallest pore size, D₁, and filter n the largest, Dₙ. The figure shows that there are no detectable aggregates above D₁ for the first half of the reaction, after which the pressure signal begins to build on filter 1. By % reaction time, aggregates of size D₂ become detectable as pressure in filter 2 begins to increase. In filter n, aggregates of size Dₙ are detectable by 9/10 the reaction time.

Particulate refers to any component in a liquid that can be trapped by a filter as the liquid containing the particulate flows through the filter (e.g. microcrystals, polymeric microgels, cross-linked polymers, latex and emulsion particles, biological cells, clusters and fibers composed of biological cells and fibers, bacteria and other microbes, cell organelle fragments, incompletely dissolved polymers, proteinacious, cellulosic and other polysaccharide particles, flocculating particles, precipitating particles, phase separating liquid systems, salt crystals, particles due to oxidation or reduction processes, and particles emanating from the reaction or process vessel itself, and aggregated therapeutic proteins). Chemically reacting refers to reactions that modify the chemical nature of the system components (e.g. formation of covalent bonds, oxidation and reduction reactions, hydrolysis, polymerization reactions, enzymatic processes, photolysis and other chemical processes driven by light, heat, catalysts, branching, grafting, and crosslinking reactions, and certain denaturation reactions). Physically reacting refers to processes in which system components associate or dissociate as particulates without changing their chemical nature (e.g. non-crosslinked microgels, microcrystallization, flocculation, coacervation, protein denaturation or renaturation, molecular conformational changes leading to associative or disintegrative reactions, micellization, formation of liposomes, emulsions, vesicles, nanostructuration or micro-structuration and self-assembly of macromolecules, nanostructuration or micro-structuraion and self-assembly of nanoparticles or microparticles). Factors that can drive physically reacting systems include temperature, dissolution in time, ultrasonication, exposure to radiation, concentration of reactants, presence of salts, acids, bases, specific ions and other agents, and changes or mixing of solvents.

Non-reactive processes refer to systems that are not reacting either chemically or physically but which release or absorb already existing particles. These include processes where polymer or natural products are shaken, stirred, agitated, strained, filtered, washed by water or other solvents, milled, or otherwise physically subjected to compressive, tensile, or shear stresses.

Filter resistance refers to the resistance of a filter in a system that allows fluid flow. ‘Filtrodynamics’ refers to the ensemble of physical devices, instruments, experimental approaches, models and theories for characterizing the filter itself and the particles using the pressure and flow sensor signals. Backpressure or pressure, when driving fluid flow, means a pressure difference across one or more filters, or across one or more points in a fluid flow path, just as ‘voltage’, when driving current flow, requires voltage differences.

A central notion of the present invention is that the characteristics of liquid flowing through filters carry important information on the size and quantity of particulates in the flow, and that proper embodiment of measurement and analysis principles related to filtered flow can furnish real-time information on processes as they occur, and also optionally allow for control of the processes.

In its essence each filter in a flow path acts as a variable resistor to the flow, whose resistance increase in time is intimately related to the nature of the filter and the concentration, size distribution, and nature of the particulates passing through it.

As particulates entrained in fluid flow through a filter capable of trapping said particulates, the pressure difference across the filter will increase and/or the flow rate through the filter will decrease. If a pump that delivers constant flow rate is used the pressure difference across the filter will increase as flow rate remains constant. If a constant pressure is used to drive the fluid flow, the flow rate will decrease as particulates build up in the filter. If a pump of intermediate characteristics is used, then there can be both a buildup or decrease of pressure difference and decrease or increase in flow rate.

The mathematical signature of how this resistance changes, in addition to the associated resistance values themselves, are directly linked to the nature, concentration, and size distribution of the particulates. The method, hence, can detect and characterize particulates, whether these are constant in a process, arise and evolve during a process, diminish during a process, or transform into other types of particulates during a process.

It is noted throughout that, whereas continuous flow operation will often be the preferred mode of operation, the principles of the present invention set forth here will also apply to monitoring situations in which intermittent flows are created in order to measure filter resistance at desired intervals. Intermittent flows can lead to longer periods of plugging-free operation of the invention. For example, in some reactions, it may be sufficient to take measurements from 1-second to 200-second flows that occur just 1-10 times per hour.

The disclosed device is much cheaper to implement than optical technologies, as it can be carried out with robust, non-sensitive pumps, filters, and pressure and flow sensors. It may require a diluted sample, and it will not normally require additional conditioning steps to obtain a measurable sample.

The disclosed devices will have applications in research and development laboratories, where new materials are discovered and developed, and reactions and processes are developed, studied and optimized. In these cases the present invention may work on reaction or process vessels that have relatively small volumes, such as fractions of a liter or just a few liters.

The disclosed devices will have applications in manufacturing plants, where products are made such as, but not limited to, paints, resins, adhesives, elastomers, synthetic rubber, pharmaceutical agents, therapeutic proteins, latex particles, emulsions, water treatment chemicals, oil recovery chemicals, pigments, metallurgical, papermaking, agricultural and food products, electronic and optical materials, composite materials, materials used as drug and vaccine delivery agents, personal care products, and machinery lubrication products.

A preferred embodiment of the present invention is a device that has at least one filter with sensors to detect pressure difference and, in some instances flow rate, of a solution in a reacting system which is then correlated to the presence, build-up, or decay of particulate populations in order to create a desired system response.

An additional embodiment is a device that has a filter with sensors to detect pressure difference or flow rate that gives a time-dependent signal of filter resistance.

An additional embodiment is a device that has a filter with sensors to detect pressure difference or flow rate that gives a time-dependent signal of particulate build-up. Based on properties of the fluid, filter resistance can be correlated to particulate build-up which can be displayed as a signal.

An additional embodiment is a device with a combination of filters in parallel and/or in series with a means of mathematical data processing and cross-correlations being made during the reaction to correlate data from sensors to particulate build-up.

An additional embodiment is a device with filters or filter arrays that can be easily cleaned in a brief backwash cycle, preparing them for further measurements in a plug-free operational mode (analogously related to defrost cycles in refrigerators and heat pumps to produce “frost-free” operation).

In an additional embodiment, in the case of filters amenable to plug-free operation, it will be possible to use backwash cycles to reset the filters for the periodic measurements during the process. In this mode of operation the pressure signal will decrease as particulates decrease.

An additional embodiment is a device with pressure and/or flow monitors that will turn off flows to given filters to prevent them from becoming plugged (again, plug-free operation). This will be especially useful for filter arrays in which the small pore size filters signal early particulate formation, and when the larger pore size filters begin to yield changing pressure and/or flows the smaller ones will have served their function and can optionally be turned off before they trap more particulates.

An additional embodiment is a corollary device to the notion of “plug-free” operation where pressure and/or flow rates through a given filter or plurality of filters in a series arrangement with multiple pressure sensors can signal when flow is switched to alternate flow paths, consisting of the same plurality of filters in a series with multiple pressure sensors which yielded the threshold changeover signal. Such an arrangement can contain at least two and also many more identical filtration flow paths in parallel in order to keep plug-free operation continuing for long periods of time without interruption. This embodiment can be used to keep a clean stream flowing to a detector train downstream from the filters. An operator or technician may be alerted to change filters.

In an additional embodiment the filter or filter arrays may be packaged in easily exchangeable cartridges, so that a technician may quickly change these out without significant operational downtime. The cartridges themselves may contain the multiple, redundant flow paths mentioned in the above “corollary”, allowing a number of plugging cycles to occur before cartridge changeover is required.

An additional embodiment is a device of pumps and filters that can be of a very small nature, at the cm or millimeter scale, with flow rates in the range of 0.001 ml/min to 100 ml/minute, for example. The whole package may, for example, fit into a box 6″ (about 15 cm) on a side.

An additional embodiment is a device that uses a direct flow from the system reactor through the filtration system.

An additional embodiment is a device that uses 2D filters, of the ‘membrane’ type. These are typically on the order of 100 μm thick and can be made of such materials as nitrocellulose, cellulose acetate, poly(vinylidene difluoride), polycarbonate, nylon, Teflon, or mixed cellulose esters.

An additional embodiment is a device that uses 3D filters that can include hollow fiber filters which can be very long, millimeters to meters, gel packed column filters (e.g. of the Gel Permeation Chromatography type), silicon bead packed columns, or sintered metal filters.

An additional embodiment is a device that may not use a pump. For example, a pump may not be necessary when the filtrodynamic motive force is provided by the pressure or the flow in a reactor.

An additional embodiment is a device that includes a pump. For example, in particular, pumps yielding flow rates in the 0.001 ml/min to 100 ml/min may be used. Types of pumps that may be used include gear pumps, a variety of piston pumps, including HPLC pumps, the eccentric cam design of Fluid Metering, Inc., peristaltic pumps, diaphragm pumps, lobe, slot, and screw pumps, or syringe pumps.

An additional embodiment is a device that uses the ACOMP technology to automatically and continuously dilute to a concentration level more amenable to not rapidly plugging the filters or requiring frequent backwash cycles.

Additional embodiments may draw directly from the reactor, use ACOMP continuous dilution, or any purpose built intermittent dilution system.

In an additional embodiment, the flow through the filter(s) can be intermittent. This can be valuable in certain cases (e.g. where filter plugging occurs quickly, time until plugging can be extended by only turning the flow through the filters on intermittently and only long enough to gather a single data point from each sensor). This will still yield the time dependence of pressure and flow rates, albeit with longer gaps between points in time. This will hence also yield filter resistances vs. time, with the same longer gaps between points in time. For example, there could be 1 to 200-second flows 1 to 10 times per hour.

In an additional embodiment, the system may be used in reversed operation, that is, when particulates diminish in size and/or concentration during a reaction (e.g. production of biofuels). In this case, the strategy may be different, in that pressure of filters that trap particles may still increase as particulates decrease in size and/or concentration, but the mathematical signature may be much different from the case of increasing particulates.

An additional embodiment is a device that involves only periodic measuring of the pressure difference and/or flow (i.e. not continuous) to keep the filters from plugging too quickly.

In an additional embodiment, the present invention can measure particle size distribution in solutions with stable particulate populations, such as end products of polymerization, or intermediate reaction aliquots which have been withdrawn and quenched. Such characterization may include determination of particle size distribution (PSD), a central focus of the particle sizing field, which is currently dominated by optically based technologies.

In an additional embodiment, the present invention characterizes particulates in a system that is not reacting physically or chemically; i.e. that is in equilibrium. For example, endproducts in solutions that contain particulates, such as polymer solutions after a reaction (or aliquots removed during the reaction) containing microgels, microcrystals, etc. will yield characteristic time-dependent filter resistance signatures Rᵢ(t) that will help characterize the particulates. This test could be performed especially rapidly on such stable products, since full strength final solutions, or solutions less dilute than would normally be used in reaction monitoring, can be used. These signatures can then be used as criteria for quickly assessing endproduct quality after a reaction.

Additional embodiments may include specific filters for filtrodynamic applications or currently existing filters. This may include filters with nominal pore sizes advertised in the sale of filters even if they seldom relate to actual uniform pore sizes on a filter. Embodiments may include fibrous meshes and 3D filters that do not have any circular pores at all, with an “effective” or “equivalent” pore size. Other embodiments may include filters, such as electron beam etched foils, that actually have uniform, well-defined pore sizes. It may be that filtrodynamics will tend towards the usage of such well-defined filters, but it will often be possible to empirically use more poorly defined pore size filters in particular settings, using flexible models.

Some embodiments will involve an array of filters and interpretations of multiple backpressure signature in time, and for some, it may suffice in some applications to use one single filter of a given pore size, for which a given amount of backpressure signals an undesirable situation.

Examples and methods of use are described herein as a basis for teaching one skilled in the art to employ the invention in any appropriate manner. These examples disclosed herein are not to be interpreted as limiting.

The following network representation is an exemplary embodiment of a means to help conceptualize design and operation of the present invention, especially in the early stages, but is not to be interpreted as limiting. Some complex filter systems and their interactions may not lend themselves readily to this representation. In any event, purely empirical models and data interpretations can be used with the present invention to achieve a desired end.

In order to assess the mathematical signature, it is convenient to depict the filter networks used in different embodiments of the invention in the language of passive resistive networks, such as in elementary electronics. In this, Ohm's law is simply V=IR, where V is voltage, I is current, and R is resistance.

In these embodiments, P=pressure (dynes/cm²) replaces voltage, Q=flow rate (cm³/s) replaces current, and R=filter resistance (g/cm⁴-s) will play an analogous role as electrical resistance (Ohms). P=QR is hence the analog to V=IR. In these embodiments, a method will experimentally measure the time dependent resistance R=R(t) for one or more filters, and from R(t), determine as much as is possible about the characteristics of the particulates in the flow and their evolution in time (or their stability in time if there is no evolution). In similar embodiments, Q may be measured in g/s, and filter resistance R may have units of 1/cm-s.

In these embodiments, measuring P(t) and Q(t) is required. P(t) may be measured by a pressure transducer, of which many types exist (e.g. SMC Corporation of America, model PSE560-01). Typical pressure ranges will be from 1 atmosphere to 10,000 atmospheres. Flow rates (Q(t)) may be measured by such means as a differential pressure transducer (e.g. Validyne Corp.), or the thermal time-of-flight device by Bronkhorst Corp. e.g. Bronkhorst Liquiflo L13. Flow rates will typically run from 0.001 ml/min up to 50 ml/min.

In similar embodiments, it is helpful to measure the microscopic form of Ohm's law and its equivalent for the Invention. J=E/ρ, where J is electrical flux (C/m²-s in MKSA units), ρ is electrical resistivity (Ω-m), and E=−∇V is the electric field (N/C), which is the gradient of the electric potential.

J=−∇P/ρ is the analogous equation for the system of the present invention, where ∇P is the pressure gradient along the flow path. This equation will be appropriate for embodiments using 3-dimensional filters, as it stands for a 3-dimensional filter of cross-sectional area A and length L. In this 3-D case R=ρL/A.

In embodiments with a 2-dimensional filter, for which one pore is plugged when a particle whose diameter is greater than the pore diameter encounters the pore, there will simply be a total penetrable cross sectional area A, the pore depth being irrelevant for changes in R(t). In this case R=ρ/A, and the pressure drop across a filter is used in J=ΔP/ρ. (Note that ∇P is the gradient of P and is not to be confused with ΔP, the difference in pressure before and after a filter).

FIG. 2 illustrates an exemplary network of n filters in parallel. In this embodiment, the time-dependent resistance of each filter is denoted R₁, R₂ . . . Rₙ. The source supplies the flow of liquid to the network, whether directly from the reactor or container vessel or by means of a pump after dilution by ACOMP or other means. In this embodiment, the pressure difference across each filter is the same and is measured by the inlet pressure sensor. This pressure may vary in time, and P(t) may be constantly recorded. The time-dependent flow rate through each filter may be measured by flow sensors Q₁, Q₂, . . . Qₙ. It is therefore possible to obtain the resistance at any moment of the filter Rᵢ(t), according to Ri(t)=P(t)/Qi(t).

With the functions Ri(t) obtained it is then possible to apply the particular filter model for the system at hand to obtain the characteristics and time evolution of the particulates. The total filter resistance of the network is given by

$$\frac{1}{{R}_{total} ( t )} = {\sum }_{i = 1}^{n} \frac{1}{Ri ( t )} ,$$

so that the total flow through the network is Q(t)=P(t)/Rₜₒₜₐₗ(t).

In some embodiments, where pressure is constant, such as from the outlet of a constant pressurized reactor, then P(t)=constant. If the source provides a constant flow Q₀, e.g. from a high pressure liquid chromatography pump, then the pressure across the system is P(t)=QₒRₜₒₜₐₗ(t).

FIG. 3 shows an exemplary series network. In this embodiment, each Ri(t) can be found according to Rᵢ(t)=ΔPᵢ(t)/Q(t), where ΔPᵢ are the pressure differences across succeeding resistors (filters), given by ΔPᵢ(t)=Pᵢ(t)−Pᵢ₊₁(t). For the n^{th} (last) filter ΔPₙ₊₁=atmospheric pressure (or the pressure of the location where the readings are taking place, which might be above or below atmospheric pressure). In many embodiments, filters in series will normally be arranged with the largest pore size first and decreasing in order of pore size to the end of the series. It is noted that if the source is of constant flow, then the single flow sensor at the entrance may be omitted, whereas if the source is of constant known pressure P₁=constant, then sensor P₁ may be omitted.

FIG. 4 illustrates exemplary filters in series, each with a pressure sensor and two-way diverter valve. In position 1, the two-way valve may direct flow into the next filter, whereas in position 2 the flow may be directed to another filter assembly, to a detector train, or to waste. In this embodiment, when the pressure drop across a filter, ΔPᵢ=Pᵢ−P_{I+1} reaches a pre-set value, the diverter valve may move to position 2, thus relieving the rest of the network upstream from that valve's backpressure. Though not shown in FIG. 4, there can be appropriate bypass lines and valves to bypass a clogged filter. The new, lowered pressure signals on the other sensors may be related to the values previous to the purge by simply adding ΔPᵢ. The controller, which actuates the valves, may monitor the pressure signals from each sensor and send out valve-actuating signals when certain pressure and pressure difference criteria are met. The controller itself may be a microcomputer, programmable logic controller, distributed control system, or any other device capable of programmable signal processing. The controller may be programmed to alert industrial operators or servomechanisms of certain conditions being reached (e.g. process completion, ready for next process step, process alert or malfunction, etc.) with lights, alarm buzzers, electronic signals to control rooms, etc. Where the controller is a microcomputer, or has sufficient onboard computing power, or outputs sensor data to a microcomputer, detailed analysis of time-dependent signals may be made with information and decision making outputs.

A similar embodiment to the above could be made that would allow for filters in series plugging in random order, instead of in a sequence from finest to coarsest as assumed in FIG. 4. In this next embodiment the diverter in position 2 would divert the flow from the first plugged filter to the next unplugged filter such that all unplugged filters would stay in operation until they are plugged in their turn.

FIG. 5 illustrates an exemplary variation that can be used if the output line is feeding a detector train rather than going to waste. In this embodiment, a three-way valve is used, where positions 1 and 2 may have the same function as in FIG. 4, and now position 3 may shut off flow through the valve. The controller may again have the same function as in the previous drawing, except that it may now command each valve into three different positions, instead of two.

Relating Time Dependent Variable Resistances, R_{I}(T), to the Nature, Concentration, Size Distribution, and Evolution of the Particulates.

To make a model connecting Rᵢ(t) to the particulate properties may require both a means of characterizing the filter itself and the particles. Below is an exemplary approach to establish an interpretive model for an ideal 2-dimensional filter. This is not to be interpreted as limiting as 3-D filters also exist, and will involve similar modeling.

Assume the filter in this embodiment to have a total active area when totally unplugged of A₀ with Mₒ pores of area aₒ; i.e. Aₒ=Mₒaₒ. In this model, it is the time-dependant reduction of penetrable area A in the filter, A(t), that leads to the increase in the resistance. That is, in R(t)=ρ/A(t), the filter resistivity ρ may be taken to be constant in the simplest model, and may be dependent only on the details of the filter and flowing liquid; viscosity of the flowing liquid, temperature, and frictional interaction between the flowing liquid and the filter material around the pores.

An unplugged filter has a ‘clean resistance’, Rₒ given by Rₒ=ρ/Aₒ, where Rₒ can be directly determined by knowledge or measurement of ΔP across the filter and Q.

Particle Characteristics.

The particle size distribution may be given by N(D,t)dD, which is the number of particles per cm³ for particles in the size range from D to D+dD at time t. An object of the invention is to determine N(D,t)dD as well as possible. This might involve determination of approximate histogram representations using a series of different size filters, such as one of the networks schematized in the figures, so that one may obtain N(Dᵢ,t)ΔDᵢ, the density of particles over the discrete range ΔDᵢ=Dᵢ−Dᵢ₊₁, where Dᵢ and Dᵢ₊₁ are the respective diameters of successive filters.

Another characteristic of the particles is how they interact with the pores in the filter. For example a microgel may stick to a pore yet not fully block it. In this case, an average fractional amount of pore area remaining after being covered by a particle can be introduced as f, and represents a ‘leaky’ plugged filter (f is a ‘leakage factor’, whose value is 0 for a non-leaky particle/pore interaction and 1 when a trapped particle does not impede flow). In this case, after all the pores are covered, microgels may continue to build up, creating a sort of percolation problem, and may lead to the asymptotic total pluggage of the filter at long enough times. The filter may also break some microgels, depending on the entanglement and strength of any given microgel. If a probability is assigned to a microgel penetrating through a pore rather than covering it in an encounter, then an exponential type of blockage function will occur. Other scenarios describing the way a given particulate reduces effective filter area can be designed as needed.

It is recalled that the particle flux J(t) is central to J(t)=ΔP/ρ, and that this flux is given, by definition, as J(t)=Q(t)n(t)/Aₒ, where n(t) is the total number of particles per cm³ at any given time that can block the filter pore size D under consideration; i.e. n(t)=∫_{D}^{∞}N(d′)dD′.

Case of Constant Flow Rate.

If the above approach is applied in an embodiment where Q(t)=Q₀=constant, and n(t)=nₒ does not vary in time, and using plugging particles that will plug a pore on contact, leaving faₒ as the remaining penetrable area of the plugged pore, then it is straightforward to show that

$$P ( t ) = \frac{{Q}_{o} {R}_{o}}{1 - {a}_{o} {J}_{o} ( 1 - f ) t} , t \leq {t}_{p} and$$

$$P ( t ) = \frac{{Q}_{o} {R}_{o}}{1 - {a}_{o} {J}_{o} ( 1 - f ) {t}_{p}} = constant for {t}_{p} .$$

where Jₒ=Qₒnₒ is a constant flux of particles, so that the filter plugs linearly in time, and is fully plugged at tₚ=1/Jₒaₒ, and Pₒ=QₒRₒ.

Another example is where particulate concentration increases in time linearly according to n(t)=βt, with Qₒ again constant. This leads to

$$P ( t ) = \frac{{Q}_{o} {R}_{o}}{( 1 - ( 1 - f ) \frac{{t}^{2}}{{t}_{p}^{2}} )} , t \leq {t}_{p}$$

$$where$$

$${t}_{p} = \sqrt{\frac{2 {N}_{o}}{β {Q}_{o}}} .$$

Another example is where the particulate concentration decreases according to n(t)=nₒ−βt, where nₒ is the initial concentration of blocking particles. The time for the particulates to disappear is t_{c}=nₒ−βt. Then

$$P ( t ) = \frac{{Q}_{o} {R}_{o}}{( {A}_{o} - ( 1 - f ) {a}_{o} {Q}_{o} ( {n}_{o} t - β {t}^{2} / 2 ) )} , t \leq {t}_{p}$$

where tₚ is found in the case where tₚ<t_{c} from solving the quadratic equation

Nₒ=Qₒ(nₒtₚ−βtₚ²/2).

As illustrated in FIG. 6, if tₚ>t_{c} then P(t) will arrive at a plateau found by substituting t_{c} for t in the expression for P(t).

FIG. 7 illustrates how it is seen from the equations for P(t) above, that 1/P(t) will yield polynomials in t that will give a more striking signature that differentiates the three scenarios: a steady particulate population yields a straight line for P(t) vs. t and the second derivative, d²P/dt²=zero (the solid line); for the case of increasing particulates d²P/dt²<0 (the dotted line); and for decreasing particulates d²P/dt²>0 (the dashed line).

FIG. 8 illustrates another set of cases under constant flow Q₀, where it is assumed that the rate of pore pluggage to degree f is proportional to the remaining amount of pore area, which yields an exponential decrease in total filter area. This scenario is expected to be encountered frequently, especially in 3D filters, and “imperfect” filters; i.e. those for which there is a probability that particles larger than the pore size can get through the filter. This is often related back to the fact that few filters are truly composed of uniform circular pores. Filters with fibrous meshes, sintered metals, porous gels, etc. have ill-defined pore sizes, and they are usually sold with a nominal or ‘effective’ pore size. Even in some cases where pores are well defined, the nature of the particulates, such as deformability, may let some particles through that are larger than the well defined pore size.

In these cases, the decrease in the number of unplugged pores is proportional to the number of remaining pores M(t) and the concentration of impinging particles N(t):

$$\frac{d M ( t )}{d t} = - p M ( t ) N ( t )$$

where p is related to the probability that a particle whose size is greater than the pore size will get trapped by the filter, the higher p is the greater the chance of particle entrapment. In the simplest case, where p and N=Nₒ are both constant, the number of remaining pores will decrease exponentially; M(t)=Mₒexp(−pNₒt).

Some examples of signatures of P(t) are seen in FIG. 8, where p is taken as constant. For N(t)=constant a non-leaky filter's (f=0) pressure may increase exponentially (dotted line). If the leakage factor f>0 then a plateau in pressure may be reached where P_{final}/Pₒ=1/f (solid line). For the case of a linearly increasing population of particles the sigmoidal shape shown below is predicted (dashed line).

Case of Constant Pressure, P₀.

FIGS. 9 and 10 shows the relationship between flow rate and time, again for an ideal 2D filter. When the concentration of particulates is constant, nₒ, it is straightforward to show that the flow rate falls off exponentially, according to Q(t)=Qₒe^{αt} where

$$α = \frac{{n}_{o} {Q}_{o}}{{M}_{o}} .$$

If the particulate concentration increases linearly in time as n(t)=βt then it can be shown that Q(t)=Qₒe^{-γt}^{²} where

$$γ = \frac{{Q}_{o} β}{2 {M}_{o}} .$$

The flow rates for these two cases are shown in FIGS. 9 and 10. FIG. 10 shows the logarithm of the reciprocal flow rate. It is revealing in that constant particle concentration yields a straight line (dotted line) and a second derivative d²Q/dt²=0, whereas a linearly increasing particulate population leads to a parabolic increase and d²Q/dt>0 (dashed line).

FIG. 11 illustrates one embodiment of the invention that enables a plugging-free operation via parallel flow paths with identical pore size filters or series of filter 1A through n XYZ (any number n of flow paths of filters with any number XYZ of filters per flow path, where preferably the filters in each path are the same as the filters in every other path—such as a series of filters have pore sizes which decrease downstream). The pressure and/or flow rates through a given filter may signal when flow is switched to alternate flow paths, headed by the same filter pore size as the path through which the same pore size filter which yielded the threshold changeover signal. A useful application of this is may be to keep a clean stream flowing to a detector train downstream from the filters. In this figure, as each successive filter path hits a predetermined pressure, the n-way fluid switching valve automatically changes flow over to the next parallel flow path with a fresh filter in it until reaching the final filter #n. Well before this happens an operator or technician may be automatically alerted to a changeover of filters or new filter cartridge unit and the technician may change out the filters or filter cartridges.

FIG. 12 shows an embodiment of the invention in which the filters are in series, each with a pressure sensor and two-way diverter valve. In position 1, the two-way valve directs flow into the next filter, whereas in position 2, it is directed to waste. In this configuration, when the pressure drop over a filter, ΔPᵢ=Pᵢ−P_{I+1} reaches a pre-set value, the diverter valve may move to position 2, thus relieving the rest of the network from that valve's backpressure. The new, lowered pressure signals on the other sensors may be related to the values previous to the changeover by simply adding ΔPᵢ. The controller, which actuates the valves, may monitor the pressure signals from each sensor and send out valve actuating signals when certain pressure and pressure difference criteria are met. The controller itself may be a microcomputer, programmable logic controller, or any other device capable of programmable signal processing. The controller may be programmed to alert industrial operators or servomechanisms of certain conditions being reached (e.g. process completion, ready for next process step, process alert or malfunction, etc.) with lights, alarm buzzers, electronic signals to control rooms, etc. Where the controller is a microcomputer, or has sufficient onboard computing power, or outputs sensor data to a microcomputer, detailed analysis of time dependent signals may be made with information and decision making outputs.

FIG. 13 illustrates an additional embodiment of the invention, in which the output line is feeding a detector train rather than going to waste. In this case, a three-way valve is used, where positions 1 and 2 may have the same function as the embodiment illustrated in FIG. 12, and now position 3 may shut off flow through the valve. The controller may again have the same function as in FIG. 12, except that it can now command each valve into three different positions, instead of two.

Putting the Invention on a Reactor.

FIGS. 14 and 15 illustrate how embodiments of the Invention, such as depicted and described above, can be used for monitoring presence and evolution of particulates in a polymerization reactor. In both diagrams, the net contents of the above type embodiments (as shown in FIGS. 2-5 and 11-13) are termed the ‘Filtrodynamic Unit’, and include filter networks, pressure and/or flow sensors, controller, and optional analysis means (such as viscosity, light scattering, turbidity, refractive index, pH, conductivity, UV/visible absorption detectors, polarimeter, IR detectors, circular dichroism, circular birefringence, fluorescence). Optionally, the control and analysis means can be integrated into the ACOMP control and analysis platform. Furthermore, the analysis results being produced in realtime may be used to control the reactor, through feedback loops controlling for example servomechanisms, temperature, reagent flows, pressure, or addition of any of the agents normally used for reaction control, or simply by alerting reactor personnel to the state of the reaction, from which they can take appropriate actions; e.g. stopping the reaction, proceeding to a subsequent stage, changing temperature, reagent feeds, quenching, or addition of any type of mentioned agents. Such agents can include, but are not limited to catalysts, initiators, monomers, comonomers, quenching agents, branching agents, cross-linking agents, salts, coagulation agents, gasses, such as air, nitrogen, or oxygen.

FIG. 14 illustrates an exemplary ‘front-end’ of an ACOMP automatic continuous dilution system. The front-end is the ensemble of pumps, mixing chambers, etc. that the ACOMP platform uses to produce a continuous diluted stream of reactor contents. This frequently includes a first stage, low pressure mixing with a continuous overflow of first dilution waste liquid. Optional placement point #1 shows where the Filtrodynamic Unit may be placed to be fed, taking advantage of this first waste stream. Optionally, an on/off fluid control valve may be used, and actuated at desired intervals by the Controller in the Filtrodynamic Unit, for intermittent “filter-saving” operation.

The optional placement point #2 illustrates the Filtrodynamic Unit as a side stream after full ACOMP dilution through the second (or multiple) stages. It can also optionally use an intermittent on/off valve.

Optional placement point #3 is directly from the ACOMP fully diluted stream. It may go to waste, or feed an ACOMP detector train (e.g. viscosity, light scattering, turbidity, refractive index, pH, conductivity, UV/visible absorption detectors, polarimeter, IR detectors, circular dichroism, circular birefringence, fluorescence) that may be optionally used for analysis of polymerization reactions.

Optional placement point #4 is directly between the reactor outlet feed and the ACOMP front-end. In this configuration the filtrodynamic unit serves to protect the ACOMP front-end from plugging due to particulates. In this configuration ‘plugging free’ operation will be used in which multiple parallel filter flow paths are used one at a time in the filtrodynamic unit to feed the ACOMP unit, the flow switching from one flow path to another when a pressure or flow signal criterion is reached. Such a plugging free operation device is shown in FIG. 11. In some instances, such as when vessel content has a dense particulate population, an optional dilution step may be introduced between the reactor outlet feed and the filtrodynamic unit. An added dilution step can also serve to extend filter life and yield longer plugging free operation periods. An important advantage of the filtrodynamic unit in optional placement point #4 is that in this configuration the filtrodynamic unit can serve to both protect the ACOMP front-end from plugging due to particulates and to characterize the particulates in the process or reaction vessel. This dual purpose use of filtrodynamics in conjunction with the ACOMP front-end is expected to also lead to much better understanding and control of reactions and processes since it will allow combining monitoring of particulate properties with the properties of the polymeric product being produced to find relationships between polymer properties and how and why particulates are formed.

An important operation in FIG. 14 is the use of two or more independent filtrodynamic units. As mentioned, for example, a filtrodynamic unit in optional placement point #4 can both protect the ACOMP system's front-end while also using plugging free operation and provide particulate monitoring and characterization. A second filtrodynamic unit before the ACOMP detectors, shown as optional placement point #3 will serve to protect the very sensitive detector train. The detector train is much more sensitive than the robust ACOMP front-end and requires higher and finer levels of filtration than the front-end. The filtrodynamic unit in placement point #3 will provide this higher level of filtration and, when combined with a device for changing among parallel flow paths, will allow for extended periods of operation in which the detectors will not foul or plug. The optical sensors, such as light scattering, refractive index, and UV/visible detectors are particularly sensitive to small amounts of particulates coating or smearing their optical components or plugging their flow paths.

FIG. 15 replaces the ACOMP front-end to achieve dilution of the reactor liquid with an intermittently operated dilution instrument. This instrument can be constructed from readily available components and is similar to ACOMP operation, except that, instead of continuous reactor withdrawal, it may make withdrawals of reactor fluid at defined time intervals, and may dilute them in a mixing chamber(s). The diluted reactor liquid may then be used to feed the Filtrodynamic Unit in defined time intervals.

FIG. 16 illustrates how an intermittent reverse flow can be used to purge and clean the filters. This can be used intermittently during a reaction, or for automatic cleaning at the end of a reaction or measurement cycle.

Determination of Particle Size Distribution (PSD).

As mentioned, the present invention not only allows monitoring changes in particulate populations during time dependent processes, such as chemical and physical reactions, it may also allow determination of the particle size distribution, PSD, itself, which is a central goal in the general field of particle sizing analysis. This sizing can be done dynamically, that is PSD can be determined as the PSD evolves in time, and also for a stable PSD.

In one embodiment, let N(D,t)dD be the number density (concentration) of particulates in the size interval D to D+dD at time t. N(D,t) is the PSD in this instance. The filtrodynamic approach may use a series of filters of discrete pore size, so that one may actually determine N(Dᵢ), or Nᵢ, which will be the concentration of particles in the size range Dᵢ to Dᵢ₊₁, where Dᵢ and Dᵢ₊₁ are the pore sizes of successive filters i and i+1. In this case, each filter blocks all particles whose diameter is greater than Dᵢ, so that each filter yields the integral of concentrations of all particles larger than its pore diameter. As used before, let n(D,t) be the concentration of all particles with sizes larger than D. n(D,t) may then related to N(D,t) by n(D,t)=∫_{D}^{∞}N(D,t)dD.

An object of particle sizing is N(D,t), so this is determined from the primary experimental data n(D,t) by differentiation

$${N ( D , t ) = \frac{d n ( {D}^{′} , t )}{d {D}^{′}} \vert}_{{D}^{′} = D} .$$

For the discrete notation required since a finite number of filters z, n(Dᵢ,t)=Σᵢ^{z}N(Dᵢ, t) may be used.

In this case, N(Dᵢ,t) is found by

$$N ( {D}_{i} , t ) = \frac{Δ n ( {D}_{i} , t )}{Δ {D}_{i}} .$$

Where Δn(Dᵢ,t)=n(Dᵢ,t)−n(Dᵢ₊₁,t), and ΔDᵢ=Dᵢ₊₁−D, (where the order is interchanged in the two definitions, since nᵢ>nᵢ₊₁ and Dᵢ₊₁>Dᵢ,) where the filters are numbered in order of increasing pore size. It is noted that it will sometimes be advantageous to use a logarithmic sequence of pore sizes, in which case the useful representation of N(Dᵢ,t) can be had as

$$N ( {D}_{i} , t ) = \frac{Δ n ( {D}_{i} , t )}{Δ log {D}_{i}} .$$

Gel Permeation Chromatography columns, for example, are used in molecular weight distribution analysis of polymers, and the columns in general separate in terms of log M, where M is polymer molecular weight.

Determination of PSD for a Stable Particle Population.

An illustration is given here of how PSD can be found for a stable population of particulates in a solution when the filtrodynamics corresponds to the ideal filter case discussed above. For this time-independent example, N(D,t)=N(D). The determination of PSD in stable solutions is a major area in particle sizing technology, so it is expected that this particular case will be developed in depth as the present invention finds increasing application.

It was shown above that reciprocal pressure in time is linear when flow rate is constant (filters in series) and PSD is constant and the filter is ideal; i.e. that it entraps all particles larger than its pore diameter with 100% efficiency until all pores are plugged. (A corresponding analysis can be made for filters in parallel). It is recalled that this model allows for leakage of the ideal filter, given by the leakage fraction f. For filter I,

$$\frac{Δ {P}_{o , i}}{Δ {P}_{i} ( t )} = 1 - {α}_{i} {J}_{o , i} ( 1 - {f}_{i} ) t , for t < {t}_{p}$$

where t_{p,i}=1/J_{0,i}aᵢ, ΔP_{o,i}=Q_{o,i}R_{o,i}, where ΔPₒ, is the pressure across filter i at t=0, J_{o,i}=nᵢQ_{0,i}/Aᵢ, nᵢ≡n(Dᵢ), aᵢ=πDᵢ²/4 is the area of a pore for filter i, and Aᵢ is the total area of the filter before any plugging occurs. ΔPᵢ(t) refers to the pressure drop across filter i; ΔPᵢ(t)=Pᵢ₋₁−Pᵢ. For the first filter i=1, Pᵢ₋₁=atmospheric pressure. If the filters are in series then Q_{o,i} is the same for all filters.

The slope of reciprocal pressure sᵢ, is

$${s}_{i} ≡ \frac{d ( \frac{Δ {P}_{o , i}}{Δ {P}_{i} ( t )} )}{d t} = - {a}_{i} {J}_{o} ( 1 - {f}_{i} ) = - \frac{{a}_{i} ( 1 - {f}_{i} ) {n}_{i} {Q}_{o , i}}{{A}_{i}} . \\ Define {α}_{i} ≡ \frac{{a}_{i} ( 1 - {f}_{i} ) {Q}_{o , i}}{{A}_{i}} .$$

Then the object of finding PSD may now be achieved by

$${n}_{i} = \frac{\vert {s}_{i} \vert}{{α}_{i}} .$$

In this example, all the parameters composing αᵢ are known or readily determinable: Q₀ is the pump flow rate, which is both known and adjustable, aᵢ and Aᵢ are known from the filter characteristics, and the leakage factor fᵢ can be determined from each filter's final pressure difference at plugging

$${f}_{i} = \frac{Δ {P}_{i} ( {t}_{p} )}{Δ {P}_{o , i}} .$$

It is noted that one could also use the plugging time tₚ, for each filter to obtain nᵢ, but there are several reasons the determination of sᵢ will generally be a superior approach. First, real filters are likely to deviate from the idealized filter here, meaning that reciprocal pressure will not be linear in time even for constant Q₀ and nᵢ. Hence tₚ will deviate from the above expression, where as an initial slope before non-ideality sets in will more accurately yield nᵢ.

Of perhaps even greater practical interest is the possibility that the slope approach could allow the PSD of multiple independent samples to be determined before plugging the filters. In principle, it might suffice to use a very short interval to determine the slope, allowing only minimal filter pluggage and re-use with other samples. The minimum time intervals for satisfactory determination of sᵢ would have to be determined experimentally.

FIGS. 17 to 19 illustrate examples of how the reciprocal pressures may lead to determination of Nᵢ.

FIG. 17 shows conceptual signals for reciprocal pressure vs. time for a series of 18 filters, where filter 1 has the smallest pore size and filter numbering increases with pore size up to filter 18, which has the largest pore size. The pluggage time tₚ for each filter is shown in the vertical lines along the time axis, and a few are labeled for illustration; e.g. t_{p,1}. The reciprocal of the plateaus value for each filter is 1/fᵢ. Different plateau values are shown to indicate that different filters may have different leakage factors fᵢ.

FIG. 18 shows the nᵢ obtained from FIG. 17, using the method just described. The absolute values of the slopes are used in computing nᵢ.

The PSD obtained from the nᵢ in FIG. 18, according to the procedure described above, is in FIG. 19. It shows the concentration (particles/cm³) in the size ranges shown on the logarithmic x-axis (microns). As in other particle sizing methodologies, the distribution below can be left as a histogram or, by well known methods, smoothed into a continuous function, fit to an assumed analytic form, etc.

In the case of non-ideal filtrodynamics, for example probabilistic filters discussed above, the corresponding pressure and flow rate signals can be used from them to obtain nᵢ. In those cases, there may not be a simple relationship, such as a linear reciprocal pressure vs. t whose slope can be used, but other characteristics of the signature can be used; e.g. for the probabilistic filter exponential rise and decay rates will furnish the information needed to obtain nᵢ.

Determining PSDS as they Change in Time.

The above approach can also be applied to dynamically evolving populations of particulates. Several approaches are available. A possible approximation that may prove quite robust is to take the pressure signals arriving from the filters during a reaction and dividing these into approximate linear elements over short time intervals. The above method can then be applied to the collection of pressure derivatives from all filters during each time period.

An example of how this approach might be implemented is seen in FIG. 20. The reciprocal pressure vs. t for 5 filters is shown, filter 1 again having the smallest pore size, and filter 5 having the largest. The vertical lines show time intervals, Δt, into which the data can be broken. Over each interval a linear fit to the reciprocal pressure of each filter can be made and the method above for determining N(Dᵢ,t) from the slopes sᵢ, applied, to yield the PSD at each time interval k; N(Dᵢ,tₖ). In the illustrative graph there are a total of 12 time intervals shown, so that 12 PSDs would be obtained from these data over the course of measurement. In FIG. 20, the dotted line segments are examples of the linear fits that would be made over a few chosen intervals Δt. According to the idealized filter model, the positive second derivative of the reciprocal pressure in later stages indicates the populations of particles whose size is comparable to those filters' pore sizes are decreasing in concentration. For filters 3 through 5 the negative second derivative in later stages indicates an increase in concentration of those larger particulate populations.

Monitoring the Buildup of Microgel Aggregates During a Polymerization Reaction.

The following is an illustrative example of particulates forming in a reaction and a specific embodiment of the present invention.
In one embodiment a polymerization reaction may be occurring in a reactor. As this reaction proceeds microgel particles may begin to form and increase in both quantity and size due to both physical microgelation and chemical polymeric cross-linking. Such reactions where this type of particle formation occurs include those involving water-soluble polymers and copolymers of acrylamide and its derivatives, vinyl pyrrolidone and its derivative, sulfonated styrene, acrylates and their derivatives, methacrylates and their derivatives, ethacrylates and their derivatives, elastomers, polyolefins, ethylene-propylene-diene rubber, styrene-butadiene rubber, high impact polystyrene, polysulfones, or polyurethanes and their copolymers.

A small stream of the reacting liquid, or a diluted stream of the reacting liquid, as provided for example by ACOMP, is pumped at constant flow rate through a group of filters in series. The backpressure on each of these filters is monitored continuously (preferably, or substantially continuously) in time. In one embodiment, the filters in series may have pore sizes that can trap particulates of the approximate sizes; 0.5 μm, 1 μm, 10 μm, 50 μm, 250 μm, and 1 mm. The order of the filters in the device is largest first in the flow stream, decreasing in order along the flow path to the smallest pore size filter at the output end. As particulates at the 0.5 μm size form and begin to be trapped in the filter the pressure on all the filters may rise equally. This signals that microgel formation at this small size range has begun. If the concentration of these particles in the flow stream increases the pressure across the filter will increase with a certain characteristic mathematical signature, which depends on how the particles are increasing the filter resistance. If the concentration increases, there will be characteristic mathematical signatures for the pressure increase corresponding to the rate of increase of concentration.

As microgels grow in size, the filters of successive pore size may each begin to build up pressures of their own with corresponding mathematical signatures that depend of the nature of the particles, each filter, and the process producing the changing particle population and characteristics. It may have been experimentally determined beforehand that a certain set of pressure signatures from the array of filters indicates certain levels of microgel size distributions and concentrations. It may have also been determined beforehand at what set of instantaneous signals the microgel content of the reactor has become undesirable and that corrective measures need to be taken.

While this system involves an array of filters and interpretations of multiple backpressure signature in time, it may suffice in some applications to use one single filter of a given pore size, for which a given amount of backpressure signals an undesirable situation.

FIG. 21 illustrates a conceptual set of pressure signals on filters 1, 2 . . . n during the hypothetical polymerization reaction. Filter 1 has the smallest pore size, D₁, and filter n the largest, Dₙ. The figure shows that there are no detectable aggregates above D₁ for the first half of the reaction, after which the pressure signal begins to build on filter 1 (solid line). By ¾ reaction time aggregates of size D₂ become detectable as pressure in filter 2 begins to increase (dotted line). In filter n, aggregates of size Dₙ are detectable by 9/10 the reaction time (dashed line). The aforementioned method of determining PSD can also be used on this type of data to obtain N(D,t), the dynamic evolution of the PSD.

Use of optical and viscosity detectors in conjunction with the basic filtrodynamic unit.

Not only is this filtrodynamic approach useful for detecting the presence and evolution of particulate populations in solutions of polymers and colloidal suspensions, it can also be used to optimize the filtration process for solutions in order to achieve the best quality solution for a given purpose and to quantify the effects of filtration on solution characteristics. This would normally be done by connecting additional detectors to the flowing system, beyond just pressure sensors and flow meters. Such detectors could include viscosity, light scattering, turbidity, refractive index, pH, conductivity, UV/visible absorption detectors, polarimeter, IR detectors, circular dichroism, circular birefringence, and fluorescence. This is not necessarily an ACOMP-based device or approach—the present invention includes apparatus which includes Filtrodynamics with one or more flow detectors, including viscosity, light scattering, pH, conductivity, turbidity, and UV/visible absorption (among others). Please note also that it would be useful to put SMSLS detectors (U.S. Pat. No. 6,618,144) on the filtrodynamic unit in some contexts.

Here are some examples:

One starts with a turbid polymeric solution that is too turbid for an optical measurement such as light scattering, RI, or polarimetry. Such a solution might be that of a natural polysaccharide that includes aggregates and microgels of polysaccharide, or bits of cellulosic or proteinaceous material, or a solution of polymers that contains physically or chemically cross-linked particles. One would then gather a series of candidate filters of different types; e.g. sintered metal frits of different porosities, Teflon, nylon, cellulose ester, poly(vinylidene difluoride) filters of different porosities and possibly different diameters (e.g. commonly used 4 mm, 13 mm and 25 mm diameters). The filters would also have appropriate inline filter holders, some of which are reusable and some of which have integral housings, for example.

The filtrodynamic arrangement then consists of one or more of these filters in series or parallel, each with a pressure sensor behind it to measure trans-filter pressure and one or more desired detectors after each filter or after the complete filtration stream. For example, turbidity is very easy and inexpensive to measure, consisting of a light source and light detector and means to read the light detector output (e.g. an A/D board in conjunction with a microcomputer), so that turbidity meters could be placed in the pre-filtered stream and after each filter to find the effect of filtration on turbidity. At the same time, the pressure sensors allow one to monitor the pressure profile on the filter to see if it is acceptable, or too steep (i.e. filter begins to plug too quickly, or runaway pressure that leads to filter pluggage or perforation), thus allowing optimization of the filtration scheme, including types and combinations of filters and flow rates.

Other detectors include using a viscometer for measuring the change in viscosity due to filtration removing viscous particles, such as crosslinked polymer, conductivity to measure the amount of charged particulates removed by filtration, polarimetry to measure the amount of a chiral molecule (e.g. polysaccharide) removed by filtration, HTDSLS light scattering, to find the density of large particles before and after filtration.

An interesting inversion of this scheme is to use the responses of the detectors, e.g. turbidity, to correlate to the time dependent pressure signals and hence calibrate the pressure signals to change in turbidity, which could be useful in online applications. Other correlations besides pressure to turbidity could include pressure to optical activity, pressure to viscosity, pressure to conductivity, pressure to molecular mass, and pressure to crosslinking.

Results of a Filtrodynamic Study

The object of these efforts was to carry out and analyze the first experiments in a recently defined field termed ‘Filtrodynamics’. The main notion of filtrodynamics is that a network of filters in series and/or parallel will act like a passive variable resistor network where each element's resistance can rise as particulates plug its pores. The time dependent signature of increasing trans-filter pressure can be related to the existence, concentration, and size distribution of particles in a flowing stream. The applications are numerous since particulates, usually unwanted, are a source of great malaise in the manufacture of everything from natural products to water purifications agents, to most polymers, and to therapeutic protein drugs. Unwanted particulates can cause reactions to fail, whole product lots to be unusable, reactors to be shut down for overhaul and maintenance, etc. The cause, existence and evolution of unwanted particulates during reactions or processing are usually unknown to a manufacturer, so that a means of monitoring their appearance and properties can be quite valuable in controlling their effects. Examples of particulates include polymer microgels, either chemically or physically linked, microcrystals, aggregates of highly cross-linked polymers, emulsified particles, clumps of associating proteins, microbes, and cellulosic debris.

When a filter perforates it is analogous to a short circuited element, whereas when a filter is fully plugged it is analogous to an open circuit condition. The time dependent signatures of filter resistance increase are monitored by pressure sensors and/or flow meters. In this work, pressure sensors are used exclusively and a high pressure liquid chromatography pump is used to deliver accurate, fixed flow rates (constant current source).

While there are many different experiments that can be performed in this as yet unexplored field, several early experimental tests were targeted:

Identification of ideal model systems; e.g. spheres that plug membranes with cylindrical pores. Latex spheres and nucleopore filters were chosen as the first candidate for an ideal system.

Testing flows in different modes: Recirculation of particle-laden liquid (analogous to a battery circuit). Flow of particle laden liquid to waste (analogous to a circuit that sinks its charge to ground). Injection of charge into a flowing stream from a hold-up loop (vaguely analogous to a photoemissive circuit element that injects charge when exposed to a pulse of light).

Identifying specific filter types for specific particulate filtering.

Exploring behavior of ‘real-world’ solutions that contain both a viscosity enhancing polymer and particulates.

Reproducibility and stochastics of repeated filtrodynamic experiments. Coupling in other detectors, besides pressure sensors, such as viscometers and concentration sensitive detectors; e.g. refractive index or UV/Visible absorption detectors.

Use of two or more filters in series.

Changing particle concentration in time.

Using mixtures of particle sizes and multiple filters.

Initial experiments focused on using 2 μm latex spheres in water as near-ideal particles for plugging filters and causing pressure increase. Many different types of filters were tried, including Nucleopore, metallic frits, PVDF, PTFE, cellulose esters, and others, in a variety of diameters, including 3 mm, 4 mm, 13 mm, and 25 mm, some in plastic housings and others in re-usable threaded filter housings. Glass spheres were also obtained to be suspended in organic solvent and used with appropriate filters.

Early Measurements Using Latex Spheres

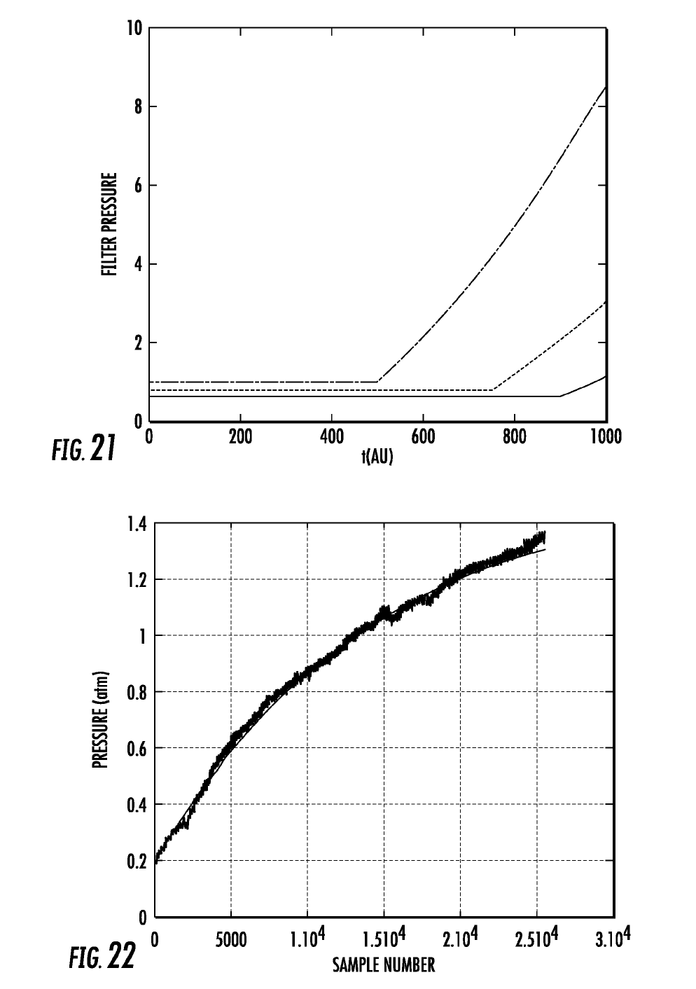

Most of these experiments were done by recirculating the sample containing the particulates continuously through the filtrodynamic circuit. Typical volumes of material were 10 ml to 50 ml and typical flow rates were 0.1 ml/min to 1.0 ml/min. FIG. 22 shows the build up of trans-filter pressure (in atmospheres) when a dilute solution of 2.0 micron latex spheres flows through a 0.45 PTFE Millipore filter of 13 mm diameter at 0.2 ml/min. It is fit very well by the equation derivable from the form presented in this application text for a probabilistic filter. This lends strong support to the possibility of a mathematical analysis of filtrodynamic time dependent signals with corresponding physical interpretation.

0.19/(1−x₁)+(x₁(x₂−1)/(−1+x₂exp(x₃(x₂−1)t))))

Characterization of Filters and Tubing for Linear Passive Resistance Properties

A basic notion of filtrodynamics is that the filters act like variable resistors to fluid flow, their resistance increasing as they begin to plug due to capturing particulates in flowing solutions. Under pure solvent flow there should be no change in resistance nor hysterisis as the current (i.e. fluid flow rate) is increased and decreased. This constituted a base level check for new filters being used. It was found that the filters used displayed constant pressures that increased linearly with flow rate and returned to their original state without hysterisis, and could be cycled again in the same way. The exception was when pressures became high enough to burst filters in a non-reversible and destructive fashion. Several membrane filters were found to burst at low pressure, such as Nucleopore.

Occasional Use of a Large Volume Injection Loop.

In some experiments the liquid containing the particulates was recirculated through the entire filtrodynamic series by a pump. In other experiments a large volume injection loop placed after the pump was used which allowed injecting these particulate containing solutions through the filters without having to pass through the pump. Some particulates led to pump plugging and so the loop method was used in some situations. This type of loop injection approach is the same as used in Gel Permeation Chromatography (often also termed Size Exclusion Chromatography), except that in this filtrodynamic approach the GPC columns are replaced by the filtrodynamic filter assemblies.

Addition of a Refractometer and Viscometer.

A Shimadzu refractometer was added into the detector train to monitor concentration of polymers and small particles. A single capillary viscometer was added to capture the profile of polymer passing through the filter as opposed to blocking particles in complex solutions, such as a natural product polysaccharide, described next. There is no limit to the number and types of detectors that can be added in series to sense the different properties of the flowing solutions. Other detectors readily added include multi-angle static light scattering (e.g. Brookhaven Instruments Corp., Holtsville, N.Y., seven angle BI-MwA scattering unit) and dynamic light scattering (e.g., Brookhaven Instruments Corp. Nano-DLS), turbidity, UV/visible absorption, fluorescence, conductivity, polarimetry, pH, and infra-red absorption.

Measurements of Natural Product Polysaccharide Solutions. (all Experiments at 0.2 ml/min Unless Otherwise Noted)

The injection loop made possible the use of highly non-ideal solutions, such as these polysaccharide solutions, which are both turbid and highly viscous. The weight average molecular weight of the polysaccharides was determined to be in excess of 10⁶ g/mole, using light scattering and also, separately, by GPC.

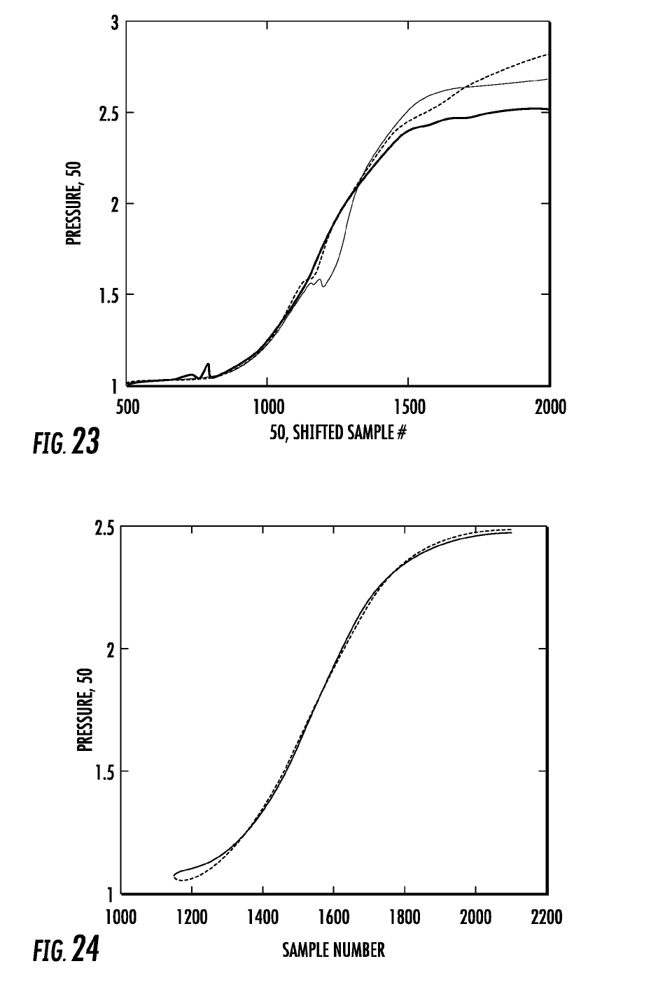

The pressure signals were shown to be quite repeatable for multiple injections of the polysaccharide solution through a fresh 0.8 μm cellulose acetate filter on three consecutive runs, as seen in FIG. 23. The units of pressure are Volts. The pressure in atmospheres is determined from this voltage by

P(atm)=(V−1)

The pressure across the filter in these experiments went from 0 to a little over 1.5 atmospheres. The time to empty the 2.5 ml loop was about 750s.

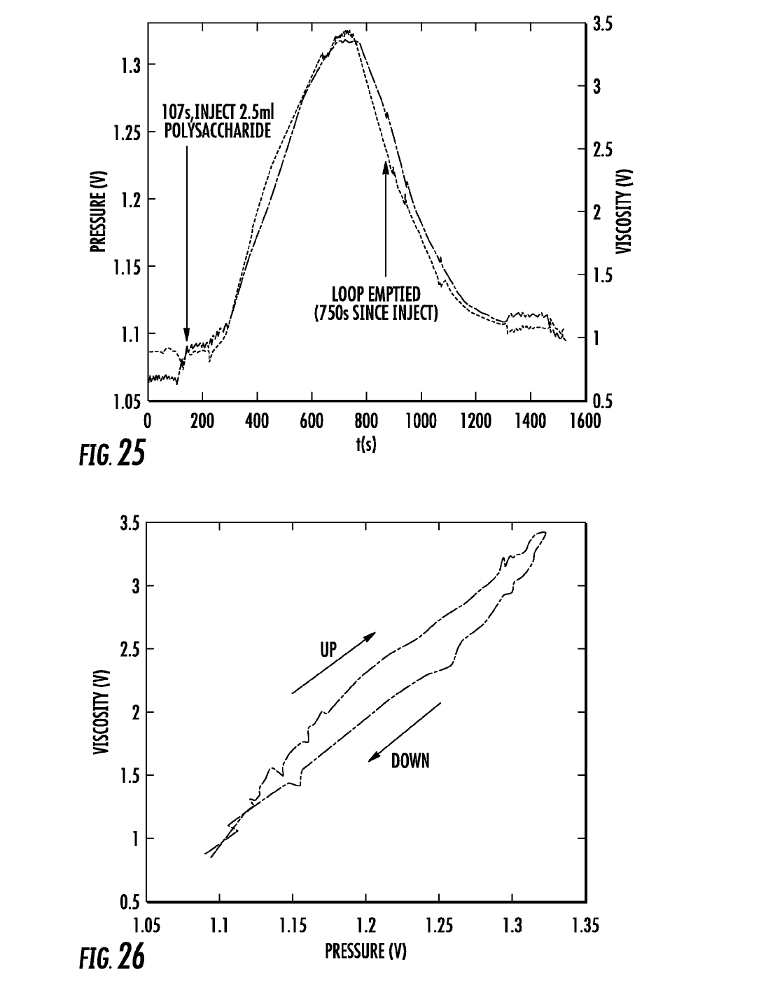

These data were also well fit by the equation form derived for a probabililstic filter, as seen in FIG. 24.

Discovery of the ‘Gatekeeping’ Effect in Viscous Flow

The first experiments, using native polysaccharide at concentrations of 3 mg/ml with metallic frit filters of different pore sizes revealed an interesting effect. Namely, the viscosity and filter pressure signals rose in unison, slowly, and then the viscosity would subside after the content of the injection loop was washed through, whereas the pressure would mostly, but not wholly subside. The behavior was surmised to be due to polymer piling up at the filter and slowly squeezing through under pressure, an effect the present inventor has termed ‘gatekeeping’. The meaning of gatekeeping is that the filter rate limits the passage of polymer chains through itself. The fact that the filter pressure only partly recovered reflected the fact that particulates in the turbid polysaccharide solution were retained on the filters.

An example of the effect is seen in FIG. 25. A 2.0 micron stainless steel frit was used in the system with a flow rate of 0.2 ml/min. The viscosity returns to very close to its original baseline at the end of the experiment, whereas the pressure ends noticeably higher than the starting value.

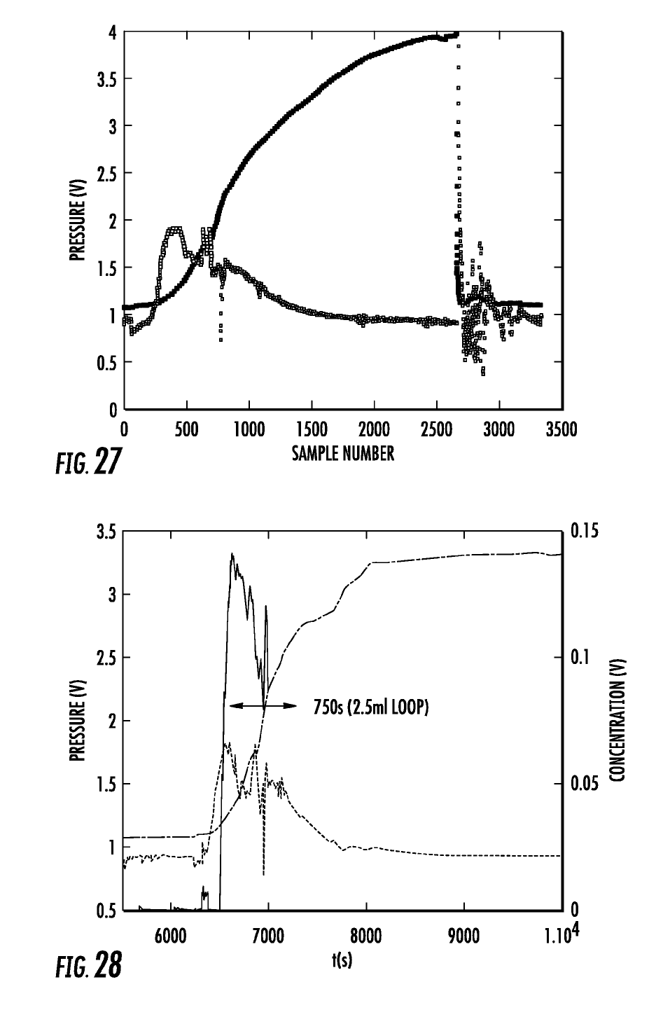

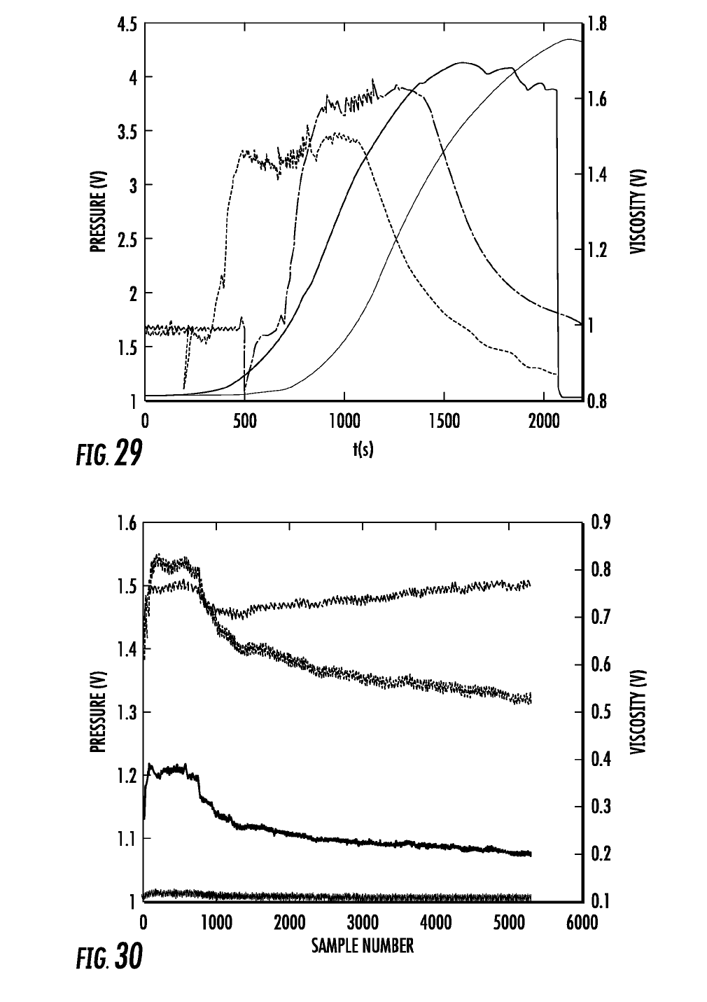

FIG. 26 shows the tight correlation between viscosity and pressure for this gatekeeping effect. The hysterisis and the fact that pressure ends somewhat higher on the return path than where it started is due to the buildup of polymer and particles on the filter.

No gatekeeping: Using a 0.8 μm cellulose ester membrane filter with the same polysaccharide solution as above yielded no gatekeeping, as seen in FIG. 27. The hallmarks of this are the very quick rise of the viscometer signal (black circles), which shows that the well dissolved polymer chains make it through the filter in a nearly square pulse with an exponential tail, without being significantly impeded by the filter, whereas the pressure signal builds up much more slowly and is due to the particulates in the polysaccharide solution gradually plugging the filter. Hence, the viscosity detects the polysaccharide chains, which are the main component of the solution, and the pressure detects the presence of the particulates, which are a minority mass fraction of the solution.

The actual amount of material in particulate vs. well dissolved polysaccharide chain form was determined by weighing the amount of material before and after filtration. In this context it was also possible to optimize filtration procedure using filtrodynamics. This involved monitoring how the pressure signal built up vs amount of polysaccharide solution flowed, and the flow rate, and coupling this with turbidity measurements of the filtrate for best optical clarification. In this case, several different filter types and pore sizes were tested before arriving at the 0.8 μm cellulose ester membrane filter as the best filtering procedure among the different filter possibilities.

Another approach to concentration determination is seen in FIG. 28. Here, a differential refractometer (RI) was used in addition to the viscometer. The RI showed, similarly to the viscometer, that the well dissolved polysaccharide chains, the majority mass fraction of the sample, passed through the filter without being delayed by the gatekeeping effect. The data illustrate both the use of multiple detectors and their ability to distinguish different aspects of the polymers and particles in the solution.

An example of a filtrodynamic experiment with two filters in series and a viscometric detector:

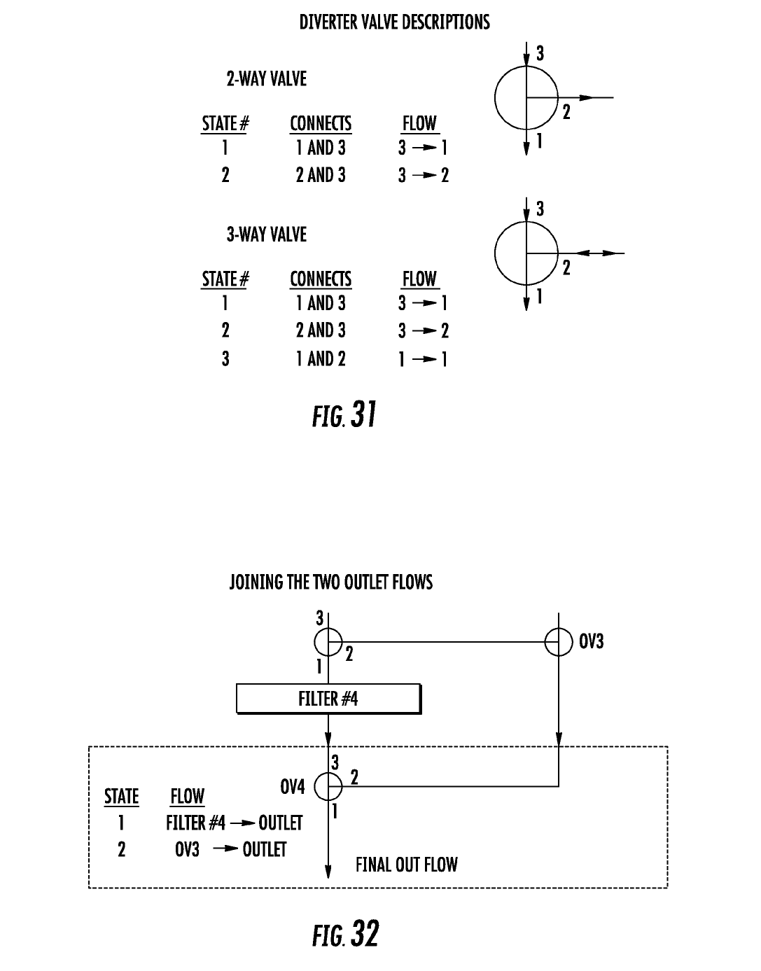

FIG. 30 shows raw data from a filtrodynamic arrangement consisting of two pressure transducers (Ashcroft Corp., type G1) measuring the transfilter pressure of two filters in series; the first was a 0.8 micron cellulose acetate filter, 13 mm diameter, and the second was a 0.45 micron PTFE Millipore membrane filter of 4 mm diameter. The particles were uniform size latex spheres of 2.0 micron diameter (Duke Scientific) in water, at 0.25 ml of stock sphere solution per ml of water, and the pump was a Shimadzu High Pressure Liquid Chromatography pump, capable of pumping constant flow rates of 0.05 ml/min to 5 ml/min up to a pressure of 100 atmospheres. The pump flow rate was 0.2 ml/min and ‘channel number’ in the graph below refers to seconds of pumping time. A total of 11 ml of sphere solution was pumped through the filters. After each filter a capillary viscometer was connected in series with the flow path to measure solution viscosity after each pumping stage. The capillary viscometer was constructed based on a differential pressure transducer by Validyne Corp, as previously described (D. P. Norwood, W. F. Reed “Comparison of Single Capillary and Bridge Viscometers as Size Exclusion Chromatography Detectors”, Int. J. Polym. Ana. and Char., 4, 99-132, 1997).

Any of the filtrodynamic units shown in for example FIG. 2-5, 11-13, 16, or 33 of the present invention can be used in conjunction with prior inventions of the present inventor shown in his prior patents and published patent applications; for example, a filtrodynamic unit can be placed:

before the light scattering detector of FIG. 16 of U.S. Pat. No. 6,653,150; before the viscosity detector of FIG. 19 of US Patent Publication No. US2004/0004717 A1; between elements 13 and 14 and/or between elements 21 and 22 of FIG. 1 of U.S. Pat. No. 7,716,969.

In addition to providing useful information about particulates, the filtrodynamic units also serve to filter out particulates which might otherwise damage downstream detectors of the apparatus in these prior patents and publications.

FIGS. 31-33 are schematics which help explain a situation in which valves in series go above their cutoff pressures in random order. When pressure exceeds the cutoff pressure, flow is diverted away from that filter.

FIG. 31 is a valve schematic.

Two-way valve positions and flows:

In position #1 the two-way valve connect ports 1 and 3 such that the flow is from 3 to 1.

In position #2 the two-way valve connect ports 2 and 3 such that the flow is from 3 to 2.

Three-way valve positions and flows:

In position #1 the three-way valve connect ports 1 and 3 such that the flow is from 3 to 1. In position #2 the three-way valve connect ports 2 and 3 such that the flow is from 3 to 2.

In position #3 the three-way valve connect ports 2 and 1 such that the flow is from 2 to 1.

FIG. 33 is an example of four filters in series that can be turned off individually as each reaches its threshold pressure in random order. The series network consists of N filters, N 3-way valves termed “Inner valves”, and N−1 two-way valves termed ‘outer valves’. Both 3-way and 2-way valves have three ports, labeled 1, 2, and 3 in the diagram. There are N pressure sensors connected to any type of computer or programmable logic controller or any device capable of reading N pressure sensor signals which are related to real or relative pressure, and sending out signals to set each of the 3-way valves in one of its 3 positions, and each of the 2-way valves in one of its 2 positions. The pressure signal read is either below a cutoff pressure (B in the table below) and flow continues through that filter, or equal to or greater than the cutoff pressure (P in the table below) at which moment flow is diverted away according to the table below. Once a filter has plugged it is no longer necessary to read its pressure, and its input value for the valve controller is locked on ‘P’. The ‘P’ values remain active until the system is reset, e.g. for a change out of filters.

Because in some states the position of one of the 3-way valves is not important, since flow is diverted around it, a value of ‘A’ is placed in the table to indicate ‘any’ position can be used. For definiteness in operation one of the three states might optionally be selected as the default for ‘A’, although this is not necessary.

Because in some states the position of one of the 2-way valves is not important, since flow is diverted around it, a value of ‘E’ is placed in the table to indicate ‘either’ position can be used. For definiteness in operation one of the two states might optionally be selected as the default for ‘E’, although this is not necessary.

The arrangement is readily extendable up to an arbitrary number of filters N, and the corresponding valve actuation diagram can be accordingly extended. It is easy to determine the valve actuation table for N>4. The valve controller will necessarily have an algorithm for generating the valve actuation states based on the series of N binary inputs (B or P).

FIG. 4 is the special case where the filters reach their pressure cutoff values from the endmost filter (normally the finest) to the first (normally the coarsest). It does not show the signals to actuate the two-way valves (as in this situation typically the last would plug first, and flow would automatically divert to waste when a filter plugs).

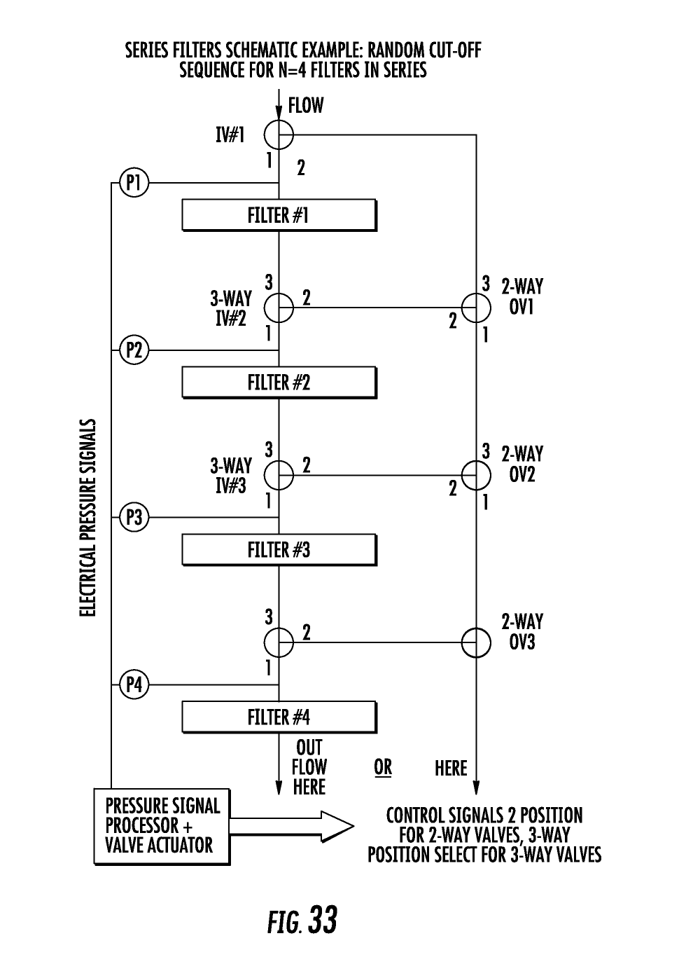

The above explanation is for the case where the filters reach their cutoff pressures in random order (see also FIG. 33 and the table below). This is a more general version of the invention but also more elaborate and expensive. FIG. 33 has a single outlet that can feed an ACOMP system or a detector train (when the two flow lines are connected with connections shown in FIG. 32). In fact, in FIG. 33 and others it should be understood that multiple Filtrodynamic units can be used, and can be used directly between a reaction or process vessel and an ACOMP or other detection system, as well as between successive dilution/conditioning stages in an ACOMP unit.

FIG. 32 shows how a single outlet flow can be had by joining the two unconnected outlets shown in the diagram with a two-way valve.

FIG. 15 has the option of adding a Filtrodynamic unit between ‘polymer reactor’ and ACOMP front-end directly. ‘Polymer reactor’ in the figures might be better termed ‘reaction or process vessel’, as often other processes than polymer reactions will be monitored.

| | | | | | | | | Two-way | | |
|---|---|---|---|---|---|---|---|---|---|---|
| Trans-filter | | | | 3-way valve | | | | valve positions | | |
| pressures | | | | positions | | | | | | OV |
| P1 | P2 | P3 | P4 | IV#1 | IV#2 | IV#3 | IV#4 | OV#1 | OV#2 | #2 |
| B | B | B | B | 1 | 1 | 1 | 1 | E | E | E |
| B | B | B | P | 1 | 1 | 1 | 2 | E | E | 2 |
| B | B | P | B | 1 | 1 | 2 | 3 | E | 2 | 2 |
| B | P | B | B | 1 | 2 | 3 | 1 | 2 | 2 | E |
| P | B | B | B | 2 | 3 | 1 | 1 | 2 | E | E |
| B | B | P | P | 1 | 1 | 2 | 3 | E | 2 | E |
| B | P | P | B | 1 | 2 | A | 3 | 2 | 1 | 2 |
| B | P | B | P | 1 | 2 | 3 | 1 | 2 | 2 | E |
| P | B | B | P | 2 | 3 | 1 | 2 | 2 | E | 3 |
| P | P | B | B | 2 | A | 3 | 1 | 1 | 2 | E |
| P | B | P | B | 2 | 3 | 2 | 3 | 2 | 2 | 2 |
| B | P | P | P | 1 | 2 | A | A | 2 | 1 | 1 |
| P | B | P | P | 2 | 3 | 2 | A | 2 | 2 | 1 |
| P | P | B | P | 2 | A | 3 | 2 | 1 | 2 | 1 |
| P | P | P | B | 2 | A | A | 3 | 1 | 1 | 2 |
| P | P | P | P | 2 | A | A | A | 1 | 1 | 1 |

B = below cutoff pressure
P = at or above cutoff pressure
A = any of 3 positions
E = either position

The current invention is aimed primarily at manufacturing industries, where the invention will be used to improve and make more efficient the manufacture of products. There is an industrial need for monitoring particulates in the polymer and natural products industries. In many chemically, biochemically, and physically reacting systems, and/or systems being non-reactively processed, certain types of particulate matter can be produced or decomposed. For example, in a polymerization reaction, particulates often form during the reaction that consist of physical microgels of the polymer being produced, or covalently cross-linked polymers, or microcrystals, or other types of aggregates, such as salt crystals. In processing natural products for example, e.g. extracting and separating polysaccharides from their precursors, microgels and ‘fines’ may be released into the process or waste streams. In general these particulates are undesirable, since they can compromise the quality of the end product; e.g. lead to irregular textures, substandard tensile strengths, difficulties in dissolution, cloudy product solutions, etc. Furthermore, the buildup of these particles can lead to fouling of large reactors which lead to costly clean-up and maintenance procedures, including reactor shutdowns and lost production time. Sometimes, however, the buildup of particulates is desirable, such as in the case of polymerization reactions whose products precipitate out of the reacting solution.

General types of reactions and processes where particulate detection is important:

A non-exhaustive list of other chemical reactions in which particulates can increase, decrease, or remain constant during reactions includes:

In many different types of polymerization reactions—those carried out in batch, in a semi-continuous or continuous way, under pressure, at ambient pressure, different temperatures, in bulk, in solvent, in multiple stages, in emulsions, inverse emulsions, micelles, suspensions, etc. particulate matter can form, such as crosslinked microgels, physically associated aggregates and microgels, microcrystals, latex particles, emulsion and oil droplets, salt crystals, coagulum, nucleated catalytic particles, oxides and debris from the reaction or processing vessel.

In emulsification of products, e.g. foodstuffs, particles can decrease in size as emulsion proceeds, and increase in size upon de-emulsification.

In bioreactors the microbial cell count can increase or decrease as a fermentation or other reaction dependent upon microbial metabolism proceeds.

In the production of biofuels particulate matter, often cellulosic, decreases in size and quantity as the raw biomass is processed into a pure fuel.

Upon blending polymeric materials micro-phase separation can occur with the concomitant production of polymeric aggregates and other particulates.

Certain multi-component systems, e.g. surfactants, metal ions and polymers in solution, will begin to aggregate and precipitate under certain environmental conditions, such as heat.

During a polymerization reaction salts or other non-polymeric species form microcrystals.

Dissolution of grains; e.g. a biopolymer, such as guar or pectin, has a broad grain size distribution, from microns to mm. As it dissolves in water the particulate population decreases.

Processing natural products—when separating out desired endproducts from intermediate products in a process stream, particulates will often be released. The particulates can be microgels of the material, undissolved fragments of cell walls and organelles, and other particulates often termed ‘fines’.

Specific types of reactions and processes where particulates are important:

The production of water-soluble polymers and copolymers in batch, semi-batch, semi-continuous, and continuous processes.

The production of water-soluble polymers and copolymers in batch, semi-batch, semi-continuous, and continuous processes that involve acrylamide.

The production of water-soluble polymers and copolymers in batch, semi-batch, semi-continuous, and continuous processes that involve copolymers of acrylamide and other comonomers.

The production of water-soluble polymers and copolymers in batch, semi-batch, semi-continuous, and continuous processes that involve copolymers of acrylamide and one or more comonomers consisting of acrylic acid, DADMAC (diallyldimethylammonium chloride), N-isopropylacrylamide, cationic derivatives of acrylamide, anionic derivatives of acrylamide, quaternized ammonium derivatives of acrylamide, Dimethylaminoethyl Acrylate (DMAEA), Dimethylaminomethyl Acrylate (DMAEMA), and styrene sulfonate.

The production of water-soluble polymers and copolymers in batch, semi-batch, semi-continuous, and continuous processes that involve copolymers of vinyl pyrrolidone and other comonomers.

The production of water-soluble polymers and copolymers in batch, semi-batch, semi-continuous, and continuous processes that involve copolymers of vinyl pyrrolidone and one or more comonomers consisting of acrylic acid, DADMAC N-isopropylacrylamide, cationic derivatives of acrylamide, anionic derivatives of acrylamide, quaternized ammonium derivatives of acrylamide, DMAEA, DMAEMA, quaternized derivative of vinyl pyrrolidone, anionic and cationic derivatives of vinyl pyrrolidone, and styrene sulfonate.

The production of polyamino acids that involve any of the naturally occurring amino acids or any derivatives of the naturally occurring amino acids.

The production of polymers based on PEG, on PEO.

Acrylates and copolymers, such as those involving acrylates, methacrylate, ethacrylates, butacrylates, and silyl based acrylates.

Styrenics and copolymers, including HIPS.

Polysulfones and other high temperature polymers.

Bis-phenol based products and copolymers.

Synthetic rubber and elastomers.

Products based on naturally occurring polysaccharides.

Use of filtrodynamics to monitor bacterial populations in fermentation reactions.

Products made from the degradation of natural products, including the following naturally occurring polysaccharides: starch, cellulose and its derivatives, lignins, pectins, xanthans, alginates, scleroglucans, hyaluronic acid and derivatives, glycosaminoglycans, gum Arabic.

Products made from urethanes, such as polyurethanes and their copolymers.

Products involving polyolefins such as polymers and copolymers of ethylene and propylene.

Products based on polymerization of silicon containing molecules.

Products made from polyamides.

Products made from polyaniline.

Processes involving the production of polyvinyl chloride and its copolymers.

Processes involving gelatin.

Other polymers, including Acrylics, Acrylonitrile-butadiene-styrenes, Alkyds, Allyls, Amino-aldehydes, Butadiene copolymers, Carbohydrate, Casein, and Cellulose acetate, Cellulose nitrates, Coal tar resins, Cresol resins, Elastomers, Epichlorohydrin diphenol, Epoxy resins, Ethylcellulose, Ethylene-vinyl acetate, Fluorohydrocarbons, Fluoro-polymers, lonomers, Isobutylene polymers, Lignins, Melamine based polymers, Methyl acrylate resins; Methyl cellulose resins; Methyl methacrylate resins, Nitrocellulose, Nylon, Phenol-formaldehyde, Polyacrylonitrile, Polyamide, Polycarbonate, Polyester, Polyethylene, Polyethylene terephathalate (PET), Polyisobutylene, Polypropylene, Polytetrafluoroethylene, Polyvinyl alcohol, Polyvinyl halide, Urea, Urea-formaldehyde, Vinyl acetate, Vinylidene, Soybean derivatives, Thermoplastics, and Thermosets.

Polymers frequently classed as synthetic rubber, including Acrylate-butadiene, Butyl, Ethylene-propylene, Chlorinated, Chloroprene rubber; Chlorosulfonated polyethylenes; Ethylene-propylene-nonconjugated diene (EPDM) rubber; Fluoro rubber; Latex, Neoprene, Nitrile, Nitrile-butadiene, Polyisobutylene, Polymethylene, Polysulfide, Silicone rubber; Stereo rubber; S-type rubber; Styrene-butadiene rubber, Styrene-chloroprene rubber; Styrene-isoprene rubber; Synthetic rubber (i.e., vulcanizable elastomers); Thermosetting vulcanizable elastomers; Thiol rubber.

Sector based products such as additive preparations for gasoline (e.g., antiknock preparations, detergents, gum inhibitors); Concrete additive preparations (e.g., curing, hardening); Drawing inks; Drilling mud compounds, conditioners, and additives; Dye preparations, clothing, fire-resistant polymers, Fire extinguisher chemical preparations; Fire retardant chemical preparations; Greases, synthetic lubricating; Inks, writing; Lubricating oils and greases, synthetic; Oil additive preparations manufacturing; Synthetic Oils, lubricating, flameproofing agents, mildewproofing agents; Rust preventive preparations.

‘Batch’ refers to reactions in which virtually all the reactants are added initially, though some reactants may be added in discrete amounts as the reaction progresses. ‘Semi-batch’ refers to reactions in which there is a continuous flow of one or more reagents into the reaction vessel for an interval of time, up to and including the entire reaction period. ‘Continuous’ refers to reactions and processes in which there is a continuous flow of reagents or products into a process stream and a continuous output of product as long as reagents are flowing into the process stream.

Vessel refers to any container, whether a vat or system of piping, for example, in which a reaction is occurring or some process is occurring. Vessel includes a batch reactor, a semi-batch reactor, a semi-continuous reactor, and a continuous reactor. A vessel can be a flow loop in which product is produced continuously.

Polymerization reaction, as used herein, includes a polymerization reaction in which polymers are created, polymer modification, and depolymerization, including degradation (such as production of biofuels). Polymer modification can include such reactions upon formed polymers as hydrolysis by acid or base, chemical and enzymatic treatments that degrade or crosslink polymer, or that functionalize with groups such as sulfates, quaternary ammonium, amino acids, polyethelyne glycol, or any other type of functional group. Depolymerization can include chemical, enzymatic, and biological (e.g. bacterial) means of reducing the molecular mass of polymers or fragments of material containing polymers, as well as physical means such as sonication, milling, ball milling, and grinding.

A vessel in which product is intentionally being produced refers to a vessel where the product being produced is a desired product for use, sale, or research purposes, and not a vessel in which the only product being produced is an undesirable by-product.

Acronyms:

2D 2-dimensional

3D 3-dimensional

ACOMP Automatic Continuous Online Monitoring of Polymerization

A/D Analog/Digital

AU Atmospheric unit

CE cellulose ester

D LS dynamic light scattering

FU Filter Unit

GPC Gel Permeation Chromatography

HEPA High-Efficiency Particle Air

HPLC high-pressure liquid chromatography

HTDSLS Heterogeneous Time Dependent Static Light Scattering

Mw molecular mass

P Pressure

PM particulate matter

PSD particle size distribution

PTFE Polytetrafluoroethylene

PVDF poly(vinylidene difluoride)

Q Flow rate

R Filter resistance

R&D Research and Development

RI refractive index/refractometer

S Source

SMSLS Simultaneous Multiple Sample Light Scattering

UV ultraviolet

1. Apparatus for monitoring particulates in a liquid flowing through a filter, comprising:
a) a correlation means for correlating measurements of the time-dependent pressure changes across the filter, or time dependent changes in flow rate of the liquid through the filter, or a combination thereof to the properties of particulates in the liquid; and
b) a pressure monitor upstream of the filter and/or a flow meter for measuring flow rate through the filter, wherein the flow rate or pressure is measured at a frequency of at least one time per hour in the liquid flowing through the filter.

2. The apparatus of claim 1, wherein the correlation means uses time-dependent pressure and/or flow rates mathematically related to properties of the particulates in the liquid.

3-16. (canceled)

17. The apparatus of claim 1, wherein the device is modular and can be easily exchanged into the flow system for quick maintenance.

18. The apparatus of claim 1, wherein the particulates are formed in a polymerization reaction.

19. (canceled)

20. The apparatus of claim 1, wherein the particulates include one or more of the following types: microcrystals; latex particles; cross-linked polymers; polymers physically associated into aggregates; chemically or physically linked polymer microgels; aggregates of highly cross-linked polymers; emulsified particles; clumps of associating proteins, microbes, cellulosic debris, latex and emulsion particles; clusters and fibers composed of biological cells and fibers; cell organelle fragments; incompletely dissolved polymers; proteinacious, cellulosic and other polysaccharide particles; flocculating particles; precipitating particles; phase separating liquid systems; salt crystals; particles due to oxidation or reduction processes; particles emanating from the reaction or process vessel itself; and, aggregated therapeutic proteins.

21-23. (canceled)

24. The apparatus of claim 1, wherein the particulate size or size distribution and/or concentration remains constant.

25. The apparatus of claim 1, wherein the particulate size or size distribution changes in time.

26. (canceled)

27. The apparatus of claim 1, wherein the particulate concentration changes in time.

28. (canceled)

29. The apparatus of claim 1, wherein two or more filters of different pore sizes, compositions, geometries, or some combination thereof are used in series in a single flow line, and wherein a backpressure across either or both filters is measured.

30. The apparatus of claim 1, wherein two or more filters of varying pore sizes, compositions, geometries, or some combination thereof are used in separate parallel flow lines and the flow rate and/or pressure through one or more of these filters is monitored.

31. The apparatus of claim 30, wherein only one flow path at a time is active and remains active until the pressure or flow rate signal on its filter reaches a criterion level at which point that path is closed and the flow diverted to the next parallel path.

32. The apparatus of claim 1, wherein filters are used in separate parallel flow lines and the flow rate through one or more of these filters is monitored, and wherein only one flow path at a time is active and remains active until the pressure signal on its filter reaches a criterion level at which point that path is closed and the flow diverted to the next parallel path.

33. The apparatus of claim 1, further comprising a means for flowing the liquid through at least one filter.

34. The apparatus of claim 1, wherein liquid is flowed through said filter intermittently.

35. The apparatus of claim 1, wherein liquid is flowed through said filter continuously.

36. The apparatus of claim 1, wherein liquid is diverted to measure as independent samples.

37. The apparatus of claim 1, further comprising a means of measuring pressure across the filter, and of flow rate of the sample through said filter.

38. The apparatus of claim 1, wherein pressure is measured and flow rate is constant.

39. The apparatus of claim 1, wherein flow rate is measured and pressure is constant.

40. The apparatus of claim 1, further comprising a means for automatically and continuously diluting the flow to any desired level of dilution.

41-42. (canceled)

43. The apparatus of claim 1, further comprising a means to divert flow through one or more alternative flow paths in parallel, each flow path with the same arrangement of one or more filters in series, when a defined pressure signal or rate of pressure change is reached in the flow path through which flow is currently occurring, so as not to flow through said flow path, and to switch flow sequentially through each alternative parallel flow paths, providing uninterrupted plug-free flow.

44. The apparatus of claim 1, further comprising an inverting valve that allows a backwash on at least one filter when a pressure or flow rate criterion is reached, to obtain a plugging-free operation of said filter.

45. The apparatus of claim 1, further comprising a pump that controls the system pressure, flow rate, or some combination thereof.

46. The apparatus of claim 1, further comprising an indication to a user of the status of the particulates in the system.

47. The apparatus of claim 1, further comprising a controller that receives information about the pressure or flow rate of the system.

48. The apparatus of claim 47, further comprising a signal sent to an operator or device regarding the particulate properties so that the operator or device can alter the course of reaction or process according to a desired path.

49. The apparatus of claim 47, further comprising a feedback loop such that information regarding the particulate properties is sent to the controller to alter the course of reaction or process according to a desired path.

50. (canceled)

51. An instrument for determining characteristics of particulates and of polymers in a solution in a vessel comprising:
a) a means for withdrawing the solution from the vessel;
b) a filter or series of filters, wherein one or more of the filters has a pressure sensor to measure the pressure before the filter and/or a flow rate sensor to measure the liquid flow rate through the filter;
c) one or more detectors that can characterize specific properties of the polymers and particulates; and,
d) a pump or pressure source to cause a liquid containing particulates and polymers to flow through the filter or series of filters and one or more detectors.

52. The instrument of claim 51, wherein the one or more detectors are placed after one or more filters, or before one or more filters, or both before and after one or more filters.

53. (canceled)

54. The instrument of claim 51, wherein the one or more detectors can characterize specific properties of particulates.

55. The device of claim 51 wherein the one or more detectors is chosen from the group consisting of viscosity, light scattering, turbidity, refractive index, pH, conductivity, UV/visible absorption detectors, polarimeter, IR detectors, circular dichroism, circular birefringence, fluorescence detectors, and Simultaneous Multiple Sample Light Scattering (SMSLS) systems.

56-57. (canceled)

58. The apparatus of claim 51, wherein the rate of automatic further comprising a correlation means for automatically, at a rate of at least one time per hour, measuring pressure detected by the pressure monitor or flow rate through the filter detected by the flow meter and correlating the pressure or flow rate to at least one of the characteristics measured by the one or more detectors.

59-62. (canceled)

63. The apparatus of claim 51, further comprising means for using the pressure or flow rate to predict time left that the filter can continue to function within specified limits of operation or plugging.

64. The apparatus of claim 51, further comprising a dilution means for diluting the solution withdrawn from the vessel.

65. The apparatus of claim 51, further comprising a correlation means for correlating measurements of the time-dependent pressure changes across the filter, or time dependent changes in flow rate of the liquid through the filter, or a combination thereof to the properties of particulates in the liquid, and a control circuit, either automatic or through a user interface, for controlling a reaction or process occurring in the vessel based on change in pressure or flow rate over time, using information obtained by the correlation means.

66. The apparatus of claim 51, further comprising a correlation means for correlating measurements of the time-dependent pressure changes across the filter, or time dependent changes in flow rate of the liquid through the filter, or a combination thereof to the properties of particulates in the liquid, and a control circuit for controlling a subsequent reaction or process based on change in pressure or flow rate over time, using information obtained by the correlation means.

67. The apparatus of claim 51, further comprising additional filters and additional sensors in parallel paths downstream of the filter of element (b), and further comprising means for automatically using information from the additional sensors to switch flow from a path in which a filter is clogged or close to being clogged to an unclogged filter which is in another path.

68. The apparatus of claim 51, wherein the means for withdrawing liquid from the vessel includes a device capable of automatically and continuously diluting and/or mixing the fluid from the vessel in one or more stages, wherein a mixing of the fluid from the vessel and a diluent occurs to create a first mixed stream, and optional subsequent mixing or mixings occur in one or more subsequent stages in which the first mixed solution is mixed with a diluent or diluents to create a mixed stream for measurement or evaluation.

69-77. (canceled)

78. The apparatus of claim 68, further comprising a primary pump and a liquid containing vessel containing high viscosity fluid, and wherein the primary pump of the device recirculates viscous liquid to the liquid containing vessel, and a fraction of this recirculating flow is diverted, either continuously, or at intervals, for mixing or diluting.

79. The apparatus of claim 78, wherein the mixing or diluting of the viscous liquid takes place in a low pressure mixing chamber.

80-81. (canceled)

82. The apparatus of claim 78, wherein the dilution factor can be held constant, or can be varied either manually or automatically during use.

83. The apparatus of claim 68, wherein a microprocessor-containing device (e.g. a microcomputer) is used to control one or more pumps, such that the dilution factor and/or detector feed flow rate can be automatically controlled.

84. The invention of claim 68, wherein the relative viscosity of the fluid changes in time.

85-94. (canceled)

95. The apparatus of claim 68, wherein the means for withdrawing liquid from the vessel and diluting the liquid comprises an extracting means for continuously extracting a first stream and a second stream of the liquid from the vessel, a first dilution/conditioning means for continually diluting and/or conditioning the first stream in one or more stages, whereby the diluted and/or conditioned first stream facilitates characterization of the dispersion of the particles, a second dilution/conditioning means for diluting and/or conditioning the second stream whereby the diluted and/or conditioned second stream facilitates characterization of the polymers, and wherein the diluted and/or conditioned first stream flows through the filter or series of filters;
and wherein the one or more detectors characterizes the polymers in the second stream.

96. The apparatus of claim 95, further comprising particle characterizing means including at least one means from the group consisting of particle size measuring means, particle size distribution determining means, average of the particle size distribution determining means, particle number density measuring means, particle chemical composition determining means, particle shape and morphology determining means, particle structure measuring means, turbidity or light scattering means for particle characterization.

97. A method for detecting and analyzing, in real-time, the presence, onset and/or evolution of particulates in a vessel in which product is being produced, comprising:
(a) withdrawing liquid from the vessel;
(b) providing a filter through which the liquid flows;
(c) providing a pressure monitor upstream of the filter and/or a flow meter for measuring flow rate through the filter.

98. The method of claim 97, wherein at least one characteristic of the particulates is measured using at least one characterizing detector from the group consisting of viscosity, light scattering, turbidity, refractive index, pH, conductivity, UV/visible absorption detectors, polarimeter, IR detectors, circular dichroism, circular birefringence, and fluorescence detectors.

99. The method of claim 98, wherein the at least one characteristic is measured automatically, at a rate of at least one time per hour, and correlating the pressure detected by the pressure monitor or flow rate through the filter detected by the flow meter with the at least one of characteristic measured.

100-102. (canceled)

103. The method of claim 97, further comprising using the pressure or flow rate to predict time left that the filter can continue to function within specified limits of plugging.

104. The method of claim 97, further comprising diluting the liquid withdrawn from the vessel.

105. The method of claim 97, further comprising controlling a reaction or process based on change in pressure or flow rate over time, using information obtained by the correlation of step (e).

106. The method of claim 97, further comprising controlling a subsequent reaction or process based on change in pressure or flow rate over time, using information obtained by the correlation of step (e).

107. The method of claim 97, wherein the step of withdrawing liquid from the vessel further comprises and diluting the liquid comprises and extracting a first stream and a second stream of the liquid from the vessel, continually diluting and/or conditioning the first stream in one or more stages, whereby the diluted and/or conditioned first stream facilitates characterization of the dispersion of the particles and flow through the filter, diluting and/or conditioning the second stream whereby the diluted and/or conditioned second stream facilitates characterization of soluble components in the liquid, and characterizing the soluble components in the second stream.

108. The method of claim 107, wherein the first and second streams are simultaneously and continuously extracted.

109-125. (canceled)

126. The method of claim 97, further comprising means for using the time-dependent data to predict time left that a filter or plurality of filters in series can continue to function within specified limits of plugging.

127-164. (canceled)

165. The apparatus of claim 1, further comprising valving and controls that will turn off flow to a given filter when a predetermined condition is met.

166. The apparatus of claim 165, wherein the predetermined condition is a pressure which is a predetermined amount above initial pressure across the filter.

167. The apparatus of claim 1, wherein the filters are packaged in easily exchangeable cartridges, so that a technician may quickly change these out without significant operational downtime.

168-173. (canceled)

174. The apparatus of claim 1, further comprising valves and a controller which actuates the valves, the controller able to monitor pressure signals and send out valve-actuating signals when certain pressure and pressure difference criteria are met.

175. The apparatus of claim 174, wherein the controller is a microcomputer, programmable logic controller, distributed control system, or any other device capable of programmable signal processing.

176. The apparatus of claim 174, wherein the controller may be programmed to alert industrial operators or servomechanisms of certain conditions being reached (e.g. process completion, ready for next process step, process alert or malfunction, etc.) with lights, alarm buzzers, electronic signals to control rooms, etc.

177. The apparatus of claim 174, wherein the controller is a microcomputer, or has sufficient onboard computing power, or outputs sensor data to a microcomputer, and detailed analysis of time-dependent signals may be made with information and decision making outputs.

178-180. (canceled)

181. The apparatus of claim 1, further comprising means for correlating time dependent pressure signals to a measured characteristic from the group consisting of turbidity, optical activity, viscosity, conductivity, molecular mass, and crosslinking, and hence calibrate the pressure signals to changes in the measured characteristic.

182-185. (canceled)

186. The method of claim 51, wherein polymer properties are predicted for a determined amount of time in advance based on information using the correlation between the pressure detected by the pressure monitor or flow rate through the filter detected by the flow meter with the one or more detectors measuring one or more characteristics of the liquid.

187. The method of claim 186, wherein the predicted polymer properties are used to control the process.

188. The method of claim 186, wherein plug rate slopes offer predictive capabilities when linked to other measures.

189. (canceled)

190. The apparatus of claim 1 whereby one or more detectors other than pressure and flow rate are used to monitor characteristics of particulates, wherein the one or more detectors is chosen from the group consisting of viscosity, light scattering, turbidity, refractive index, pH, conductivity, UV/visible absorption detectors, polarimeter, IR detectors, circular dichroism, circular birefringence, and fluorescence detectors.

191. The apparatus of claim 190 whereby signals from the one or more detectors are correlated with signals from the pressure and/or flow rate monitors to obtain information on the particulates.

192. The apparatus of claim 1, further comprising means for using the pressure or flow rate to predict time left that the filter can continue to function within specified limits of plugging.

193. The method of claim 97, whereby the time dependent signals from the pressure and/or flow sensors are used to mathematically determine properties of the particulates.

194. The method of claim 97, whereby the time dependent signals from the pressure and/or flow sensors are used to mathematically monitor the presence, onset, and/or evolution of particulates in the liquid.

195. The apparatus of claim 1 wherein a plurality of pressure and/or flow sensors are used with a plurality of filters.

196. The method of claim 97 in which the pressure across the filter or flow rate through the filter is correlated to properties of the particles causing pressure and/or flow rate changes.
